(19) United States
(12) Patent Application Publication
(10) Pub. No.: US 2021/0374222 A1
(43) Pub. Date: Dec. 2, 2021

(54) DETECTION SYSTEM AND METHOD FOR AUTHENTICATION

(71) Applicant: Japan Display Inc., Tokyo (JP)

(72) Inventors: Masahiro TADA, Minato-ku (JP); Takashi NAKAMURA, Minato-ku (JP); Akio TAKIMOTO, Minato-ku (JP)

(73) Assignee: Japan Display Inc., Tokyo (JP)

(21) Appl. No.: 17/444,913

(22) Filed: Aug. 12, 2021

Related U.S. Application Data
(63) Continuation of application No. PCT/JP2019/044970, filed on Nov. 15, 2019.

(30) Foreign Application Priority Data
Feb. 19, 2019 (JP) 2019-027836

(51) Int. Cl. G06F 21/32; G06F 21/35; A61B 5/1172; A61B 5/00; G06K 9/00

(52) U.S. Cl. CPC G06F 21/32; G06F 21/35; A61B 5/1172; G06K 2009/00932; A61B 5/6898; G06K 9/00087; G06K 2009/0006; A61B 5/681

(57) ABSTRACT

A detection system includes a portable object that comprises a sensor configured to detect biological information on a user and is portable by the user, and a function execution device configured to acquire the biological information on the user detected by the sensor through communication, and execute a predetermined function based on an authentication result of the user associated with the biological information on the user.

### CROSS-REFERENCE TO RELATED APPLICATION

This application is a continuation of PCT international application No. PCT/JP2019/044970 filed on Nov. 15, 2019, which designates the United States, incorporated herein by reference, and which claims the benefit of priority from Japanese Patent Application No. 2019-027836 filed on Feb. 19, 2019, incorporated herein by reference.

### BACKGROUND

### 1. Technical Field

The present disclosure relates to a detection system and a method for authentication.

### 2. Description of the Related Art

In some cases, biological information is used to perform user authentication. For example, Japanese Patent Application Laid-open Publication No. 2011-113112 (JP-A-2011-113112) describes an entrance management apparatus that detects the biological information to perform entrance management.

However, according to JP-A-2011-113112, a user needs to perform an operation to be authenticated to the entrance management apparatus in order to enter a room, and thus, may need to spend time and effort for the authentication. Not only for the entrance management, the time and effort for the authentication are required to be reduced.

The present disclosure has been made in view of the above-described problem, and aims to provide a detection system and a method for authentication capable of reducing the time and effort for the authentication.

### SUMMARY

A detection system according to an embodiment of the present disclosure includes a portable object that comprises a sensor configured to detect biological information on a user and is portable by the user, and a function execution device configured to acquire the biological information on the user detected by the sensor through communication, and execute a predetermined function based on an authentication result of the user associated with the biological information on the user.

A method for authentication according to an embodiment of the present disclosure includes a biological information acquiring step of acquiring biological information on a user through communication from a sensor that is provided in a portable object portable by the user, and is configured to detect the biological information on the user, and a function executing step of executing a predetermined function based on an authentication result of the user associated with the biological information on the user.

### BRIEF DESCRIPTION OF THE DRAWINGS

FIG. 1 is a schematic diagram illustrating a detection system according to a first embodiment of the present disclosure;

FIG. 2 is an exemplary portable terminal according to the first embodiment;

FIG. 3 is a sectional view illustrating a schematic sectional configuration of the portable terminal according to the first embodiment;

FIG. 4 is a block diagram illustrating a configuration example of the portable terminal according to the first embodiment;

FIG. 5 is a circuit diagram of the portable terminal;

FIG. 6 is an equivalent circuit diagram illustrating a partial detection area;

FIG. 7 is a timing waveform diagram illustrating an operation example of a biological information detection device;

FIG. 8 is a plan view schematically illustrating the partial detection area of a sensor according to the first embodiment;

FIG. 9 is a IX-IX sectional view of FIG. 8;

FIG. 10 is a graph schematically illustrating a relation between a wavelength and an optical absorption coefficient of each of a first photodiode and a second photodiode;

FIG. 11 is an equivalent circuit diagram illustrating the partial detection area according to another example;

FIG. 12 is a schematic sectional view of the partial detection area according to the other example;

FIG. 13 is a sectional view illustrating a schematic sectional configuration of a switching element included in a drive circuit;

FIG. 14 is a block diagram illustrating functional configurations of a function execution device and the portable terminal according to the first embodiment;

FIG. 15 is a flowchart explaining authentication processing according to the first embodiment;

FIG. 16 is a diagram illustrating another example of the portable terminal;

FIG. 17 is a schematic diagram of a detection system according to a second embodiment of the present disclosure;

FIG. 18 is a flowchart explaining authentication processing according to the second embodiment;

FIG. 19 is a schematic diagram of a detection system according to a third embodiment of the present disclosure;

FIG. 20 is a schematic diagram illustrating an exemplary state where a card is inserted in a function execution device; and

FIG. 21 is a flowchart explaining authentication processing according to the third embodiment.

### DETAILED DESCRIPTION

The following describes embodiments for carrying out the present disclosure in detail with reference to the drawings. The present disclosure is not limited to the description of the embodiments given below. Components to be described below include those easily conceivable by those skilled in the art or those substantially identical thereto. Moreover, the components to be described below can be appropriately combined. What is disclosed herein is merely an example, and the present disclosure naturally encompasses appropriate modifications easily conceivable by those skilled in the art while maintaining the gist of the disclosure. To further clarify the description, the drawings schematically illustrate, for example, widths, thicknesses, and shapes of various parts as compared with actual aspects thereof, in some cases. However, they are merely examples, and interpretation of the present disclosure is not limited thereto. The same element as that illustrated in a drawing that has already been discussed is denoted by the same reference numeral through the description and the drawings, and detailed description thereof will not be repeated in some cases where appropriate.

### First Embodiment

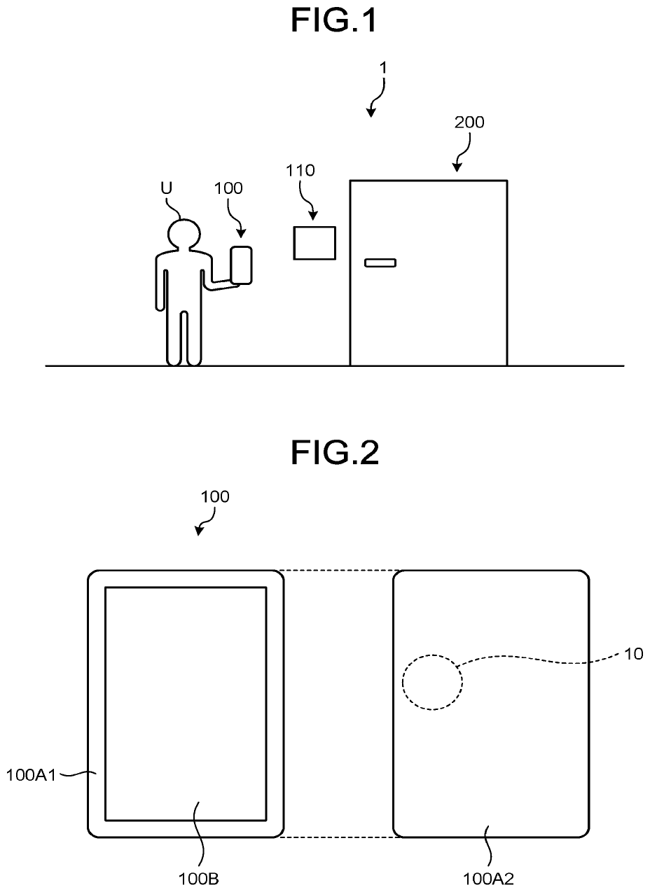

Overall Configuration of Detection System FIG. 1 is a is a schematic diagram illustrating a detection system according to a first embodiment of the present disclosure. As illustrating in FIG. 1, a detection system 1 according to the first embodiment includes a portable terminal 100, a function execution device 110, and a device to be operated 200. The detection system 1 is a system in which the function execution device 110 acquires biological information on a user U from the portable terminal 100, and executes a predetermined function based on the acquired biological information.

FIG. 2 is an example of the portable terminal according to the first embodiment. The portable terminal 100 serving as a portable object is portable by the user U, and includes a sensor 10 for detecting the biological information on the user U. The portable terminal 100 according to the first embodiment is a terminal that can be carried and operated by the user, and is herein, for example, a smartphone or a tablet computer. In the example of FIG. 2, the portable terminal 100 has, on a front face 100A1 thereof, a display area 100B for displaying an image and receiving operations of the user. The portable terminal 100 includes the sensor 10 on a back face 100A2 that is a surface on the opposite side of the front face 100A1. The portable terminal 100 is, however, not limited to the smartphone or the tablet computer, and only needs to be an object portable by the user U. The sensor 10 may be provided in any position, not limited to be provided on the back face 100A2.

The function execution device 110 illustrated in FIG. 1 acquires the biological information on the user U from the sensor 10 through communication. If the user U has been authenticated in an authentication result of the user U associated with the biological information on the user U, the function execution device 110 executes the predetermined function. In the example of FIG. 1, the function execution device 110 is an entrance management apparatus for managing entrance of the user U. In the example of FIG. 1, the device to be operated 200 is a door provided with an electronic key. In the example of FIG. 1, the function execution device 110 performs authentication, based on the biological information on the user U, on whether the user U can enter a room, and if the user U has been authenticated, that is, if the user U is allowed to enter the room, operates the device to be operated 200 to unlock the electronic key of the device to be operated 200 to bring the user U into a state of being allowed to enter the room. If, instead, the user U cannot be authenticated, that is, if the user U is not allowed to enter the room, the function execution device 110 keeps the electronic key of the device to be operated 200 locked to bring the user U into a state of not being allowed to enter the room. That is, in the example of FIG. 1, the predetermined function is a function to operate the device to be operated 200. However, the predetermined function is not limited to the function to operate the device to be operated 200, and may be any function that is set in advance to be executed by the function execution device 110. A detailed configuration of the function execution device 110, for example, will be described later.

Configuration of Portable Terminal

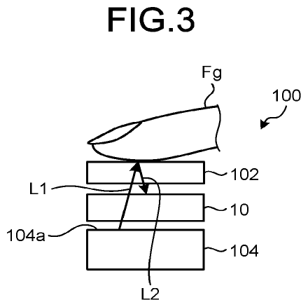

A configuration of the portable terminal 100 including the sensor 10 will be described. FIG. 3 is a sectional view illustrating a schematic sectional configuration of the portable terminal according to the first embodiment. FIG. 3 illustrates a stacking configuration of a cover glass 102, the sensor 10, and a light source unit 104. The cover glass 102, the sensor 10, and the light source unit 104 are provided by being stacked in this order.

The light source unit 104 has a light-emitting surface 104a for emitting light, and emits light L1 from the light-emitting surface 104a toward the sensor 10. The light source unit 104 is a backlight. The light source unit 104 may include, for example, light-emitting diodes (LEDs) for emitting light in a predetermined color as a light source. The light source unit 104 may be what is called a side light-type backlight that includes a light guide plate provided in a position corresponding to the sensor 10 and a plurality of light sources arranged at one end or both ends of the light guide plate. The light source unit 104 may be what is called a directly below-type backlight that includes the light sources (such as the LEDs) provided directly below the sensor 10. The light source unit 104 is not limited to the backlight. The light source unit 104 may be provided on a lateral side or an upper side of the portable terminal 100, and may emit the light L1 from the lateral side or the upper side of a finger Fg of the user.

The sensor 10 is provided so as to face the light-emitting surface 104a of the light source unit 104. In other words, the sensor 10 is provided between the light source unit 104 and the cover glass 102. The light L1 emitted from the light source unit 104 passes through the sensor 10 and the cover glass 102. The sensor 10 is, for example, a light reflective biological information sensor, and can detect asperities (such as a fingerprint) on a surface of the finger Fg or a palm by detecting light L2 reflected on an interface between the cover glass 102 and air. The sensor 10 may detect the light L2 reflected in the finger Fg or the palm to detect a vascular pattern or to detect other biological information. The color of the light L1 from the light source unit 104 may be changed depending on the detection target. For example, in the case of the fingerprint detection, the light source unit 104 can emit the blue or green light L1, and in the case of the vascular pattern detection, the light source unit 104 can emit the infrared light L1.

The cover glass 102 is a member for protecting the sensor 10 and the light source unit 104, and covers the sensor 10 and the light source unit 104. The cover glass 102 is, for example, a glass substrate. The cover glass 102 is not limited to the glass substrate, and may be, for example, a resin substrate. The cover glass 102 need not be provided. In this case, a protection layer is provided on the surface of the portable terminal 100, and the finger Fg makes contact with the protection layer of the portable terminal 100.

The portable terminal 100 may be provided with a display panel instead of the light source unit 104. The display panel may be, for example, an organic electroluminescent (EL) (organic light-emitting diode (OLED)) display panel or an inorganic EL (μ-LED or mini-LED) display. Alternatively, the display panel may be a liquid crystal display (LCD) panel that uses liquid crystal elements as display elements, or an electrophoretic display (EPD) panel that uses electrophoretic elements as the display elements. Even in this case, display light emitted from the display panel passes through the sensor 10, and the fingerprint of the finger Fg and the biological information can be detected based on the light L2 reflected by the finger Fg.

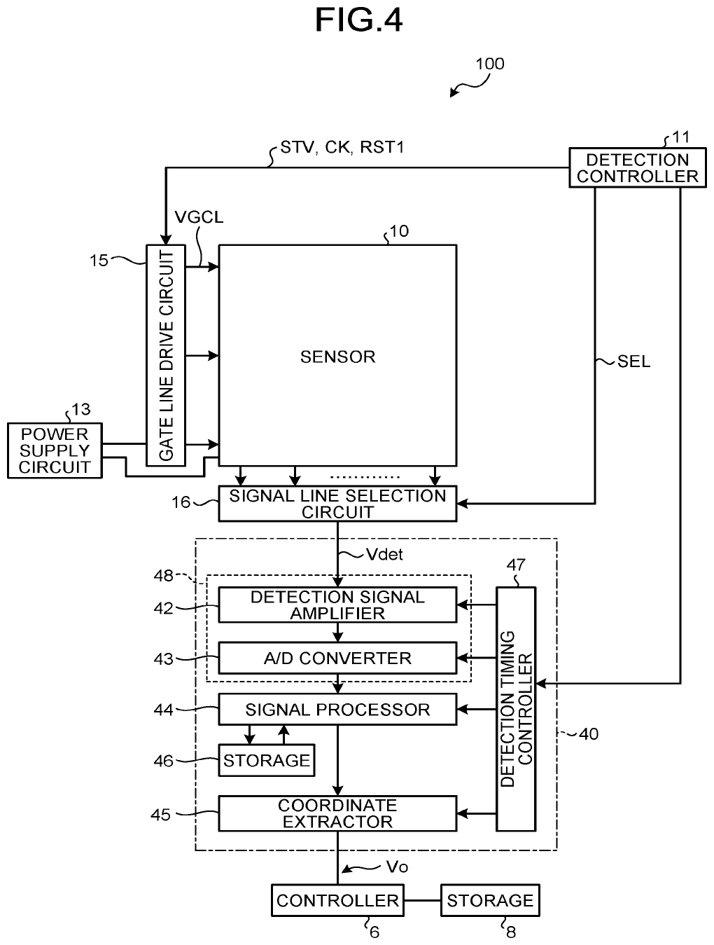

FIG. 4 is a block diagram illustrating a configuration example of the portable terminal according to the first embodiment. As illustrated in FIG. 4, the portable terminal 100 includes a controller 6, a storage 8, the sensor 10, a detection controller 11, a power supply circuit 13, a gate line drive circuit 15, a signal line selection circuit 16, and a detector 40. The controller 6 is an arithmetic device, that is, a central processing unit (CPU) mounted on the portable terminal 100. The controller 6 performs various types of processing, for example, by reading a computer program from the storage 8. The storage 8 is a memory for storing therein, for example, content of arithmetic operations and information on the computer program of the controller 6, and includes at least one of external storage devices such as a random-access memory (RAM), a read-only memory (ROM), and a hard disk drive (HDD).

The sensor 10 is an optical sensor including first and second photodiodes PD1 and PD2 that serve as photoelectric conversion elements. Each of the first and the second photodiodes PD1 and PD2 included in the sensor 10 outputs an electrical signal corresponding to light emitted thereto as a detection signal Vdet to the signal line selection circuit 16. The sensor 10 performs the detection in response to a gate drive signal VGCL supplied from the gate line drive circuit 15.

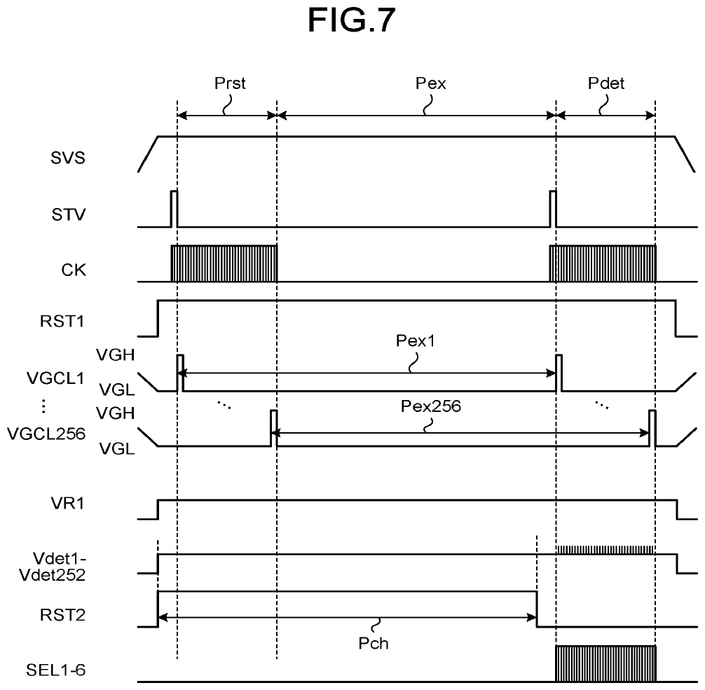

The detection controller 11 is a circuit that supplies respective control signals to the gate line drive circuit 15, the signal line selection circuit 16, and the detector 40 to control operations thereof. The detection controller 11 supplies various control signals including, for example, a start signal STV, a clock signal CK, and a reset signal RST1 to the gate line drive circuit 15. The detection controller 11 also supplies various control signals including, for example, a selection signal SEL to the signal line selection circuit 16. The power supply circuit 13 is a circuit provided in the portable terminal 100, and supplies voltage signals including, for example, a power supply signal SVS (refer to FIG. 7) to, for example, the sensor 10 and the gate line drive circuit 15.

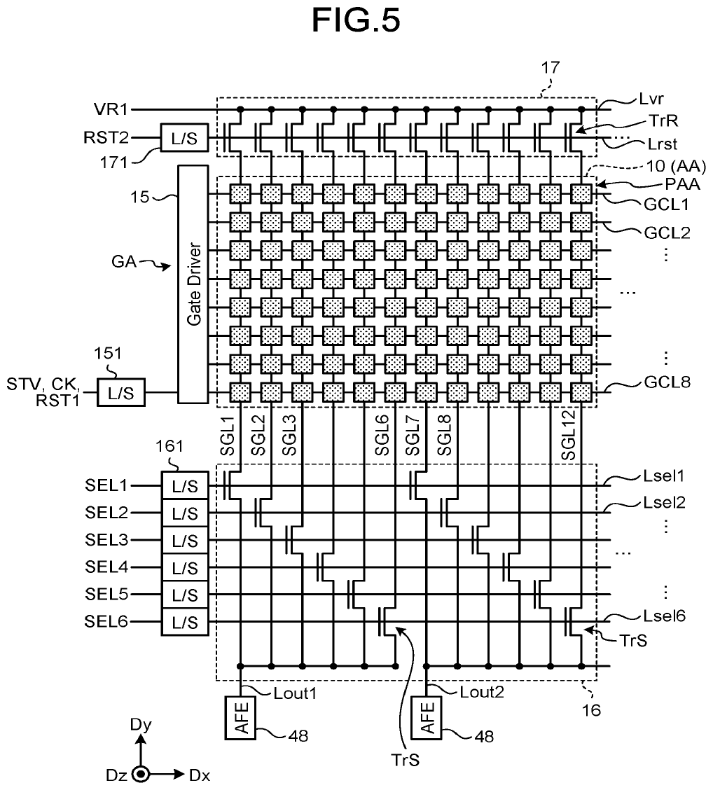

The gate line drive circuit 15 is a circuit that drives a plurality of gate lines GCL (refer to FIG. 5) based on the various control signals. The gate line drive circuit 15 sequentially or simultaneously selects the gate lines GCL, and supplies the gate drive signals VGCL to the selected gate lines GCL. Through this operation, the gate line drive circuit 15 selects the first and the second photodiodes PD1 and PD2 coupled to the gate lines GCL.

The signal line selection circuit 16 is a switch circuit that sequentially or simultaneously selects a plurality of signal lines SGL (refer to FIG. 5). The signal line selection circuit 16 couples the selected signal lines SGL to an analog front-end circuit (AFE) 48 described below serving as a detection circuit based on the selection signal SEL supplied from the detection controller 11. Through this operation, the signal line selection circuit 16 outputs the detection signal Vdet of each of the first and the second photodiodes PD1 and PD2 to the detector 40. The signal line selection circuit 16 is, for example, a multiplexer.

The detector 40 is a circuit that includes the AFE 48, a signal processor 44, a coordinate extractor 45, a storage 46, and a detection timing controller 47. The detection timing controller 47 controls, based on a control signal supplied from the detection controller 11, the AFE 48, the signal processor 44, and the coordinate extractor 45 so as to operate in synchronization with one another.

The AFE 48 is a signal processing circuit having functions of at least a detection signal amplifier 42 and an analog-to-digital (A/D) converter 43. The detection signal amplifier 42 amplifies the detection signal Vdet output from the sensor 10 through the signal line selection circuit 16. The A/D converter 43 converts the analog signal output from the detection signal amplifier 42, that is, the amplified detection signal Vdet into a digital signal.

The signal processor 44 is a logic circuit that detects a predetermined physical quantity received by the sensor 10 based on the output signal of the AFE 48, that is, the digitalized detection signal Vdet. When the finger Fg or the palm is in contact with or close to the cover glass 102 overlapping the sensor 10, the signal processor 44 can detect the asperities (that is the fingerprint) of the surface of the finger Fg or the vascular pattern of the finger Fg or the palm based on the detection signal Vdet from the AFE 48. Hereinafter, unless otherwise noted, the term “proximity” refers to the case where the finger Fg or the palm is in contact with the cover glass 102 overlapping the sensor 10, or the finger Fg or the palm is in a position close to the cover glass 102 to such an extent that the biological information is detectable.

The storage 46 temporarily stores therein a signal calculated by the signal processor 44. The storage 46 may be, for example, a random-access memory (RAM) or a register circuit.

The coordinate extractor 45 is a logic circuit that obtains the detected coordinates of the asperities of the surface of, for example, the finger Fg when the proximity of the finger Fg or the palm is detected by the signal processor 44. The coordinate extractor 45 combines the detection signals Vdet output from the first and the second photodiodes PD1 and PD2 of the sensor 10 to generate two-dimensional information representing a shape of the asperities (that is, the fingerprint) of the surface of the finger Fg and a shape of the vascular pattern of the finger Fg or the palm. This two-dimensional information can be said as the biological information on the user. The coordinate extractor 45 may output the detection signals Vdet as sensor outputs Vo, without calculating the detected coordinates. In this case, the detection signals Vdet may be called the biological information on the user.

The controller 6 acquires the two-dimensional information created by the coordinate extractor 45, that is, the biological information on the user detected by the sensor 10.

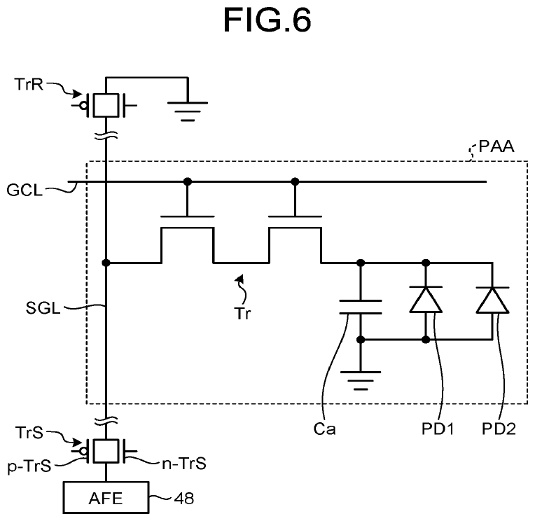

The following describes a circuit configuration example and an operation example of the portable terminal 100. FIG. 5 is a circuit diagram of the portable terminal. FIG. 6 is an equivalent circuit diagram illustrating a partial detection area. FIG. 7 is 4 a timing waveform diagram illustrating an operation example of a biological information detection device.

As illustrated in FIG. 5, the sensor 10 has a plurality of partial detection areas PAA arranged in a matrix having a row-column configuration. As illustrated in FIG. 6, each of the partial detection areas PAA includes the first and the second photodiodes PD1 and PD2, a capacitive element Ca, and a first switching element Tr. The first switching element Tr is provided correspondingly to the first and the second photodiodes PD1 and PD2. The first switching element Tr is constituted by a thin-film transistor, and in this example, constituted by an re-channel thin-film transistor (TFT).

The gates of the first switching element Tr are coupled to each of the gate lines GCL. The source of the first switching element Tr is coupled to each of the signal lines SGL. The drain of the first switching element Tr is coupled to a cathode electrode 34 of a corresponding one of the first photodiodes PD1, a cathode electrode 54 of a corresponding one of the second photodiodes PD2, and one end of the capacitive element Ca. An anode electrode 35 of the first photodiode PD1, an anode electrode 55 of the second photodiode PD2, and the other end of the capacitive element Ca are coupled to a reference potential, for example, a ground potential. In this way, the first and the second photodiodes PD1 and PD2 are coupled in parallel in the same direction to the first switching element Tr.

A third switching element TrS and a fourth switching element TrR are coupled to the signal line SGL. The third switching element TrS and the fourth switching element TrR are elements included in a drive circuit that drives the first switching element Tr. In the present embodiment, the drive circuit includes, for example, the gate line drive circuit 15, the signal line selection circuit 16, and a reset circuit 17 that are provided in a peripheral area GA. The third switching element TrS is constituted by, for example, a complementary metal-oxide semiconductor (CMOS) transistor obtained by combining a p-channel transistor p-TrS with an n-channel transistor n-TrS. In the same manner, the fourth switching element TrR is constituted by a CMOS transistor.

When the fourth switching element TrR of the reset circuit 17 is turned on, the capacitive element Ca is supplied with a reference signal VR1 serving as an initial potential of the capacitive element Ca from the power supply circuit 13. This operation resets the capacitive element Ca. When the partial detection area PAA is irradiated with light, a current corresponding to an amount of the light flows through each of the first and the second photodiodes PD1 and PD2. As a result, an electrical charge is stored in the capacitive element Ca. After the first switching element Tr is turned on, a current corresponding to the electrical charge stored in the capacitive element Ca flows through the signal line SGL. The signal line SGL is coupled to the AFE 48 through the third switching element TrS of the signal line selection circuit 16. Thus, the portable terminal 100 can detect a signal corresponding to the amount of the light emitted to the first and the second photodiodes PD1 and PD2 for each of the partial detection areas PAA.

As illustrated in FIG. 5, the gate lines GCL extend in a first direction Dx, and are coupled to the partial detection areas PAA arranged in the first direction Dx. A plurality of gate lines GCL1, GCL2, . . . , GCL8 are arranged in a second direction Dy, and are each coupled to the gate line drive circuit 15. In the following description, the gate lines GCL1, GCL2, . . . , GCL8 will each be simply referred to as the gate line GCL when need not be distinguished from one another. Although the number of the gate lines GCL is eight, this is merely an example. Eight or more, such as 256, of the gate lines GCL may be arranged.

The first direction Dx is a direction in a plane parallel to an insulating substrate 21, and is, for example, a direction parallel to the gate lines GCL. The second direction Dy is a direction in a plane parallel to the insulating substrate 21, and is, for example, a direction orthogonal to the first direction Dx. The second direction Dy may intersect the first direction Dx without being orthogonal thereto. A third direction Dz is a direction orthogonal to the first direction Dx and the second direction Dy, and is a direction orthogonal to the insulating substrate 21.

The signal lines SGL extend in the second direction Dy, and are coupled to the partial detection areas PAA arranged in the second direction Dy. A plurality of signal lines SGL1, SGL2, . . . , SGL12 are arranged in the first direction Dx, and are each coupled to the signal line selection circuit 16 and the reset circuit 17. Although the number of the signal lines SGL is 12, this is merely an example. Twelve or more, such as 252, of the signal lines SGL may be arranged. In FIG. 5, the sensor 10 is provided between the signal line selection circuit 16 and the reset circuit 17. The present disclosure is not limited thereto. The signal line selection circuit 16 and the reset circuit 17 may be coupled to the same ends of the signal lines SGL.

The gate line drive circuit 15 receives the various control signals such as the start signal STV, the clock signal CK, and the reset signal RST1 through a level shifter 151. The gate line drive circuit 15 includes a plurality of second switching elements TrG (not illustrated). The gate line drive circuit 15 sequentially selects the gate lines GCL1, GCL2, . . . , GCL8 in a time-division manner through operations of the second switching elements TrG. The gate line drive circuit 15 supplies the gate drive signal VGCL through a selected one of the gate lines GCL to corresponding ones of the first switching elements Tr. This operation selects the partial detection areas PAA arranged in the first direction Dx as the detection targets.

The signal line selection circuit 16 includes a plurality of selection signal lines Lse1, a plurality of output signal lines Lout, and the third switching elements TrS. The third switching elements TrS are provided correspondingly to the respective signal lines SGL. Six of the signal lines SGL1, SGL2, . . . , SGL6 are coupled to a common output signal line Lout1. Six of the signal lines SGL7, SGL8, . . . , SGL12 are coupled to a common output signal line Lout2. The output signal lines Lout1 and Lout2 are each coupled to the AFE 48.

The signal lines SGL1, SGL2, . . . , SGL6 are grouped into a first signal line block, and the signal lines SGL7, SGL8, . . . , SGL12 are grouped into a second signal line block. The selection signal lines Lse1 are coupled to the gates of the respective third switching elements TrS included in one of the signal line blocks. One of the selection signal lines Lse1 is coupled to the gates of the third switching elements TrS in the signal line blocks. Specifically, selection signal lines Lsel1, Lsel2, . . . , Lsel6 are coupled to the third switching elements TrS corresponding to the signal lines SGL1, SGL2, . . . , SGL6. The selection signal line Lsel1 is coupled to one of the third switching elements TrS corresponding to the signal line SGL1 and one of the third switching elements TrS corresponding to the signal line SGL7. The selection signal line Lsel2 is coupled to one of the third switching elements TrS corresponding to the signal line SGL2 and one of the third switching elements TrS corresponding to the signal line SGL8.

The detection controller 11 (refer to FIG. 4) sequentially supplies the selection signals SEL to the selection signal lines Lse1 through level shifters 161. This operation causes the signal line selection circuit 16 to operate the third switching elements TrS to sequentially select the signal lines SGL in one of the signal line blocks in a time-division manner. The signal line selection circuit 16 simultaneously selects one of the signal lines SGL in each of the signal line blocks. With the above-described configuration, the portable terminal 100 can reduce the number of integrated circuits (ICs) including the AFE 48 or the number of terminals of the ICs.

As illustrated in FIG. 5, the reset circuit 17 includes a reference signal line Lvr, a reset signal line Lrst, and the fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The detection controller 11 (refer to FIG. 4) supplies a reset signal RST2 to the reset signal line Lrst through a level shifter 171. This operation turns on the fourth switching elements TrR to electrically couple the signal lines SGL to the reference signal line Lvr. The power supply circuit 13 (refer to FIG. 4) supplies the reference signal VR1 to the reference signal line Lvr. This operation supplies the reference signal VR1 to the capacitive elements Ca included in the partial detection areas PAA.

The following describes the operation example of the portable terminal 100. As illustrated in FIG. 7, the portable terminal 100 includes a reset period Prst, an exposure period Pex, and a reading period Pdet. The power supply circuit 13 supplies the power supply signal SVS to the first and the second photodiodes PD1 and PD2 through the reset period Prst, the exposure period Pex, and the reading period Pdet. The detection controller 11 supplies the reference signal VR1 and the reset signal RST2 serving as high-level voltage signals to the reset circuit 17 from a time before the reset period Prst starts. The detection controller 11 supplies the start signal STV to the gate line drive circuit 15, and thus, the reset period Prst starts.

During the reset period Prst, the gate line drive circuit 15 sequentially selects the gate line GCL based on the start signal STV, the clock signal CK, and the reset signal RST1. The gate line drive circuit 15 sequentially supplies the gate drive signal VGCL to the gate line GCL. The gate drive signal VGCL has a pulsed waveform having a high-level voltage VGH and a low-level voltage VGL. In FIG. 6, 256 of the gate lines GCL are provided, and gate drive signals VGCL1, . . . , VGCL256 are sequentially supplied to the gate lines GCL.

Thus, during the reset period Prst, the capacitive elements Ca of all the partial detection areas PAA are sequentially electrically coupled to the signal lines SGL, and are supplied with the reference signal VR1. As a result, capacities of the capacitive elements Ca are reset.

After the gate drive signal VGCL256 is supplied to the gate line GCL, the exposure period Pex starts. The start timing and end timing of actual exposure periods Pex1, . . . , Pex256 in the partial detection areas PAA corresponding to the respective gate lines GCL differ from one another. Each of the exposure periods Pex1, . . . , Pex256 starts at a time when the gate drive signal VGCL changes from the high-level voltage VGH to the low-level voltage VGL during the reset period Prst. Each of the exposure periods Pex1, . . . , Pex256 ends at a time when the gate drive signal VGCL changes from the low-level voltage VGL to the high-level voltage VGH during the reading period Pdet. The lengths of exposure time of the exposure periods Pex1, . . . , Pex256 are equal.

During the exposure period Pex, the current corresponding to the light emitted to the first and the second photodiodes PD1 and PD2 flows in each of the partial detection areas PAA. As a result, the electrical charge is stored in each of the capacitive elements Ca.

At a time before the reading period Pdet starts, the detection controller 11 sets the reset signal RST2 to a low-level voltage. This operation stops the reset circuit 17 operating. During the reading period Pdet, the gate line drive circuit 15 sequentially supplies the gate drive signals VGCL1, . . . , VGCL256 to the gate lines GCL in the same manner as during the reset period Prst.

For example, during a period in which the gate drive signal VGCL1 is at the high-level voltage VGH, the detection controller 11 sequentially supplies selection signals SEL1, . . . , SEL6 to the signal line selection circuit 16. This operation sequentially or simultaneously couples the signal lines SGL for the partial detection areas PAA selected by the gate drive signal VGCL1 to the AFE 48. As a result, the detection signal Vdet is supplied to the AFE 48. In the same manner, the signal line selection circuit 16 sequentially selects the signal line SGL in each period in which a corresponding one of the gate drive signals VGCL is set to the high-level voltage VGH. Thus, the portable terminal 100 can output the detection signals Vdet of all the partial detection areas PAA to the AFE 48 during the reading period Pdet.

The portable terminal 100 may perform the detection by repeatedly performing the processing during the reset period Prst, the exposure period Pex, and the reading period Pdet. Alternatively, the portable terminal 100 may start the detection operation when having detected that the finger Fg, for example, is in proximity to the portable terminal 100.

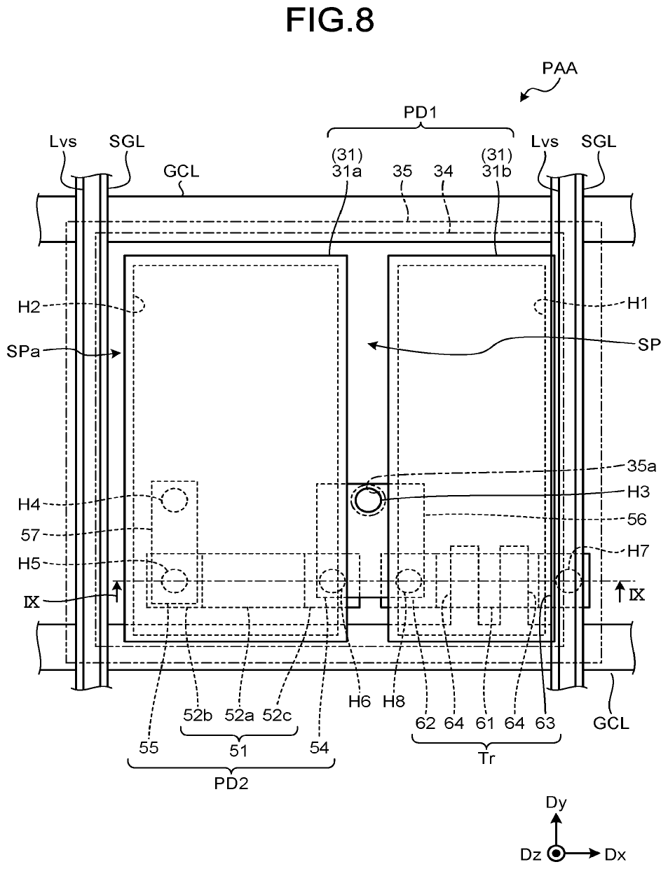

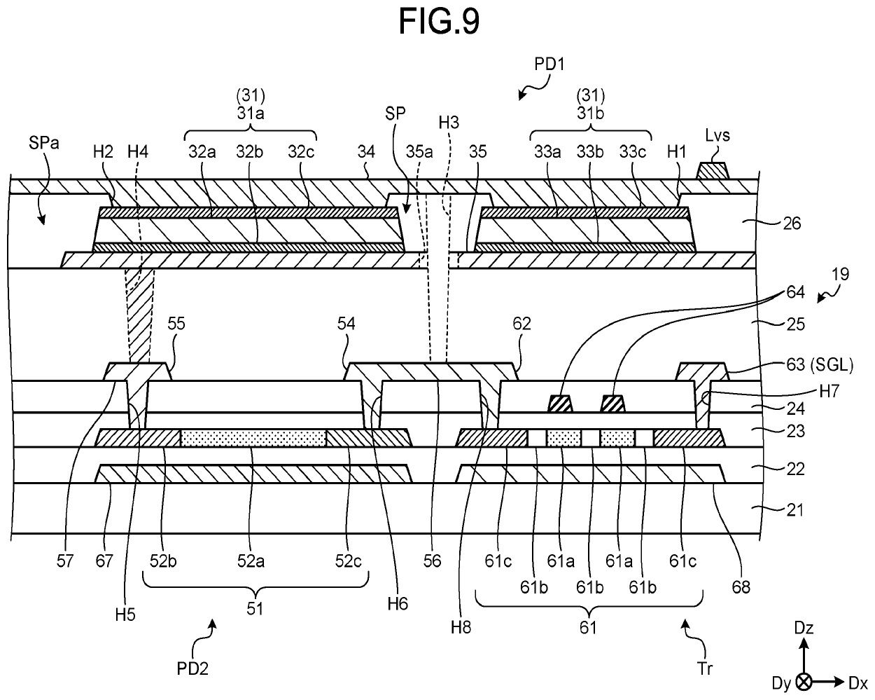

The following describes a detailed configuration of the sensor 10. FIG. 8 is a plan view schematically illustrating the partial detection area of the sensor according to the first embodiment. FIG. 9 is a IX-IX sectional view of FIG. 8. For ease of viewing, FIG. 8 illustrates the cathode electrode 34 and the anode electrode 35 with long dashed double-short dashed lines.

In the following description, in a direction orthogonal to a surface of the insulating substrate 21, a direction from the insulating substrate 21 toward the first photodiode PD1 will be referred to as the “upper side” or simply as “above”, and a direction from the first photodiode PD1 toward the insulating substrate 21 will be referred to as the “lower side” or simply as “below”. The term “plan view” refers to a case of viewing from the direction orthogonal to the surface of the insulating substrate 21.

As illustrated in FIG. 8, the partial detection area PAA of the sensor 10 is an area surrounded by the gate lines GCL and the signal lines SGL. The first photodiode PD1, the second photodiode PD2, and the first switching element Tr are provided in the partial detection area PAA, that is, in the area surrounded by the gate lines GCL and the signal lines SGL. Each of the first and the second photodiodes PD1 and PD2 is, for example, a positive-intrinsic-negative (PIN) photodiode.

The first photodiode PD1 includes a first semiconductor layer 31, the cathode electrode 34, and the anode electrode 35. The first semiconductor layer 31 includes a first partial semiconductor layer 31a and a second partial semiconductor layer 31b. The first and the second partial semiconductor layers 31a and 31b of the first photodiode PD1 are of amorphous silicon (a-Si). The first and the second partial semiconductor layers 31a and 31b are provided adjacent to each other with a space SP provided therebetween in the first direction Dx. The cathode electrode 34 and the anode electrode 35 are continuously provided over an area overlapping the first partial semiconductor layer 31a, the second partial semiconductor layer 31b, and the space SP. In the following description, the first and the second partial semiconductor layers 31a and 31b may each be simply referred to as the first semiconductor layer 31 when need not be distinguished from one another.

The first photodiode PD1 is provided so as to overlap the second photodiode PD2. Specifically, the first partial semiconductor layer 31a of the first photodiode PD1 overlaps the second photodiode PD2. The second photodiode PD2 includes a second semiconductor layer 51, the cathode electrode 54, and the anode electrode 55. The second semiconductor layer 51 is of polysilicon. The second semiconductor layer 51 is more preferably of low-temperature polysilicon (hereinafter, referred to as low-temperature polycrystalline silicon (LTPS)).

The second semiconductor layer 51 has an i region 52a, a p region 52b, and an n region 52c. The i region 52a is disposed between the p region 52b and the n region 52c in plan view. Specifically, the p region 52b, the i region 52a, and the n region 52c are arranged in this order in the first direction Dx. The polysilicon of the n region 52c is doped with impurities to form an n+ region. The polysilicon of the p region 52b is doped with impurities to form a p+ region. The i region 52a is, for example, a non-doped intrinsic semiconductor, and has lower conductivity than those of the p region 52b and the n region 52c.

The second semiconductor layer 51 is coupled to the first partial semiconductor layer 31a of the first photodiode PD1 through a first relay electrode 56 and a second relay electrode 57. In the present embodiment, a portion of the first relay electrode 56 overlapping the second semiconductor layer 51 serves as the cathode electrode 54, and a portion of the second relay electrode 57 overlapping the second semiconductor layer 51 serves as the anode electrode 55. A detailed coupling configuration between the second semiconductor layer 51 and the first photodiode PD1 will be described later.

The first switching element Tr is provided in an area overlapping the second partial semiconductor layer 31b of the first photodiode PD1. The first switching element Tr includes a third semiconductor layer 61, a source electrode 62, a drain electrode 63, and gate electrodes 64. The third semiconductor layer 61 is of polysilicon in the same manner as the second semiconductor layer 51. The third semiconductor layer 61 is more preferably of LTPS.

In the present embodiment, a portion of the first relay electrode 56 overlapping the third semiconductor layer 61 serves as the source electrode 62, and a portion of the signal line SGL overlapping the third semiconductor layer 61 serves as the drain electrode 63. The gate electrodes 64 branch in the second direction Dy from the gate line GCL, and overlap the third semiconductor layer 61. In the present embodiment, the two gate electrodes 64 are provided so as to overlap the third semiconductor layer 61 to form what is called a double-gate structure.

The first switching element Tr is coupled to the cathode electrode 34 of the first photodiode PD1 and the cathode electrode 54 of the second photodiode PD2 through the first relay electrode 56. The first switching element Tr is also coupled to the signal line SGL.

More specifically, the first switching element Tr is provided on the insulating substrate 21 as illustrated in FIG. 9. The insulating substrate 21 is, for example, a light-transmitting glass substrate. The insulating substrate 21 may alternatively be a resin substrate or a resin film formed of a light-transmitting resin such as polyimide. In the portable terminal 100, the first photodiode PD1, the second photodiode PD2, and the first switching element Tr are formed above the insulating substrate 21. This configuration allows the portable terminal 100 to have an area of a detection area AA larger than that in a case of using a semiconductor substrate such as a silicon substrate.

Light-blocking layers 67 and 68 are provided above the insulating substrate 21. An undercoat film 22 is provided above the insulating substrate 21 so as to cover the light-blocking layers 67 and 68. The undercoat film 22, a gate insulating film 23, and a first interlayer insulating film 24 are inorganic insulating films, and are formed using, for example, a silicon oxide (SiO) film, a silicon nitride (SiN) film, or a silicon oxynitride (SiON) film. Each of the inorganic insulating films is not limited to a single layer, but may be a laminated film.

The second semiconductor layer 51 and the third semiconductor layer 61 are provided above the undercoat film 22. That is, the second semiconductor layer 51 of the second photodiode PD2 and the third semiconductor layer 61 of the first switching element Tr are provided in the same layer. The light-blocking layer 67 is provided between the second semiconductor layer 51 and the insulating substrate 21 in the third direction Dz. This configuration can restrain the light L1 from directly irradiating the second photodiode PD2. The light-blocking layer 68 is provided between the third semiconductor layer 61 and the insulating substrate 21 in the third direction Dz. This configuration can reduce a light leakage current of the first switching element Tr.

The third semiconductor layer 61 includes i regions 61a, lightly doped drain (LDD) regions 61b, and n regions 61c. The i regions 61a are formed in areas overlapping the respective gate electrodes 64. The n regions 61c are high-concentration impurity regions that are formed in areas coupled to the source electrode 62 and the drain electrode 63. The LDD regions 61b are low-concentration impurity regions that are formed between the n regions 61c and the i regions 61a and between the two i regions 61a.

The gate insulating film 23 is provided above the undercoat film 22 so as to cover the second semiconductor layer 51 and the third semiconductor layer 61. The gate electrodes 64 are provided above the gate insulating film 23. That is, the first switching element Tr has what is called a top-gate structure in which the gate electrodes 64 are provided on the upper side of the third semiconductor layer 61. However, the first switching element Tr may have what is called a dual-gate structure in which the gate electrodes 64 are provided on both the upper side and the lower side of the third semiconductor layer 61, or may have a bottom-gate structure in which the gate electrodes 64 are provided on the lower side of the third semiconductor layer 61.

The first interlayer insulating film 24 is provided above the gate insulating film 23 so as to cover the gate electrodes 64. The first interlayer insulating film 24 is also provided on the upper side of the second semiconductor layer 51. The first relay electrode 56, the second relay electrode 57, and the signal line SGL are provided above the first interlayer insulating film 24. In the first switching element Tr, the source electrode 62 (first relay electrode 56) is coupled to the third semiconductor layer 61 through a contact hole H8, and the drain electrode 63 (signal line SGL) is coupled to the third semiconductor layer 61 through a contact hole H7.

In the second photodiode PD2, the cathode electrode 54 (first relay electrode 56) is coupled to the n region 52c of the second semiconductor layer 51 through a contact hole H6. This configuration couples the cathode electrode 54 of the second photodiode PD2 to the first switching element Tr. The anode electrode 55 (second relay electrode 57) is coupled to the p region 52b of the second semiconductor layer 51 through a contact hole H5.

A second interlayer insulating film 25 is provided above the first interlayer insulating film 24 so as to cover the second photodiode PD2 and the first switching element Tr. The second interlayer insulating film 25 is an organic film, and is a planarizing film that planarizes asperities formed by various conductive layers. The second interlayer insulating film 25 may be formed of one of the above-mentioned inorganic materials.

The anode electrode 35 of the first photodiode PD1 is provided above the second interlayer insulating film 25 of a backplane 19. The anode electrode 35, the first and the second partial semiconductor layers 31a and 31b, and the cathode electrode 34 are stacked in this order to form the first photodiode PD1. The backplane 19 is a drive circuit board that drives the sensor on a per predetermined detection area basis. The backplane 19 includes the insulating substrate 21, and the first switching elements Tr, the second switching elements TrG, various types of wiring, and so forth provided on the insulating substrate 21.

The first partial semiconductor layer 31a includes an i-type semiconductor layer 32a, a p-type semiconductor layer 32b, and an n-type semiconductor layer 32c. The second partial semiconductor layer 31b includes an i-type semiconductor layer 33a, a p-type semiconductor layer 33b, and an n-type semiconductor layer 33c. The i-type semiconductor layers 32a, 33a, the p-type semiconductor layers 32b, 33b, and the n-type semiconductor layers 32c, 33c are specific examples of the photoelectric conversion elements. In FIG. 9, the i-type semiconductor layers 32a, 33a are provided between the p-type semiconductor layers 32b, 33b and the n-type semiconductor layers 32c, 33c in the direction (third direction Dz) orthogonal to the surface of the insulating substrate 21. In the present embodiment, the p-type semiconductor layers 32b, 33b, the i-type semiconductor layers 32a, 33a, and the n-type semiconductor layers 32c, 33c are stacked in this order above the anode electrode 35.

In the n-type semiconductor layers 32c, 33c, a-Si is doped with impurities to form the n+ regions. In the p-type semiconductor layers 32b, 33b, a-Si is doped with impurities to form the p+ regions. The i-type semiconductor layers 32a, 33a are, for example, non-doped intrinsic semiconductors, and have lower conductivity than those of the n-type semiconductor layers 32c, 33c and the p-type semiconductor layers 32b, 33b.

The cathode electrode 34 and the anode electrode 35 are of a light-transmitting conductive material such as indium tin oxide (ITO). The cathode electrode 34 is an electrode for supplying the power supply signal SVS to the photoelectric conversion layer. The anode electrode 35 is an electrode for reading the detection signal Vdet.

The anode electrode 35 is provided above the second interlayer insulating film 25. The anode electrode 35 is continuously provided across the first and the second partial semiconductor layers 31a and 31b. The anode electrode 35 is coupled to the second relay electrode 57 through a contact hole H4 provided in the second interlayer insulating film 25.

A third interlayer insulating film 26 is provided so as to cover the first and the second partial semiconductor layers 31a and 31b. The third interlayer insulating film 26 is an organic film, and is a planarizing film that planarizes asperities formed by the first and the second partial semiconductor layers 31a and 31b. The cathode electrode 34 is provided above the third interlayer insulating film 26. The cathode electrode 34 is continuously provided above the first and the second partial semiconductor layers 31a and 31b. The cathode electrode 34 is coupled to the first and the second partial semiconductor layers 31a and 31b through contact holes H2 and H1 provided in the third interlayer insulating film 26. With this configuration, the first and the second partial semiconductor layers 31a and 31b are coupled in parallel between the anode electrode 35 and the cathode electrode 34, and serve as one photoelectric conversion element.

The cathode electrode 34 is coupled to the first relay electrode 56 through a contact hole H3 in the space SP between the first and the second partial semiconductor layers 31a and 31b. The contact hole H3 is a through-hole passing through the second interlayer insulating film 25 and the third interlayer insulating film 26 in the third direction Dz. An opening 35a is provided at a portion of the anode electrode 35 overlapping the contact hole H3, and the contact hole H3 is formed through the opening 35a. With the above-described configuration, the cathode electrode 34 of the first photodiode PD1 and the cathode electrode 54 of the second photodiode PD2 are coupled to the first switching element Tr through the first relay electrode 56. In addition, the anode electrode 35 of the first photodiode PD1 is couple to the anode electrode 55 of the second photodiode PD2 through the second relay electrode 57.

The capacity of the capacitive element Ca illustrated in FIG. 6 is provided in the space SP located between the anode electrode 55 and the cathode electrode 34 facing each other with the third interlayer insulating film 26 interposed therebetween, or is provided in a space SPa at the periphery of the first photodiode PD1 located between the anode electrode 55 and the cathode electrode 34 facing each other with the third interlayer insulating film 26 interposed therebetween. The capacitive element Ca stores therein a positive electrical charge during the exposure period Pex.

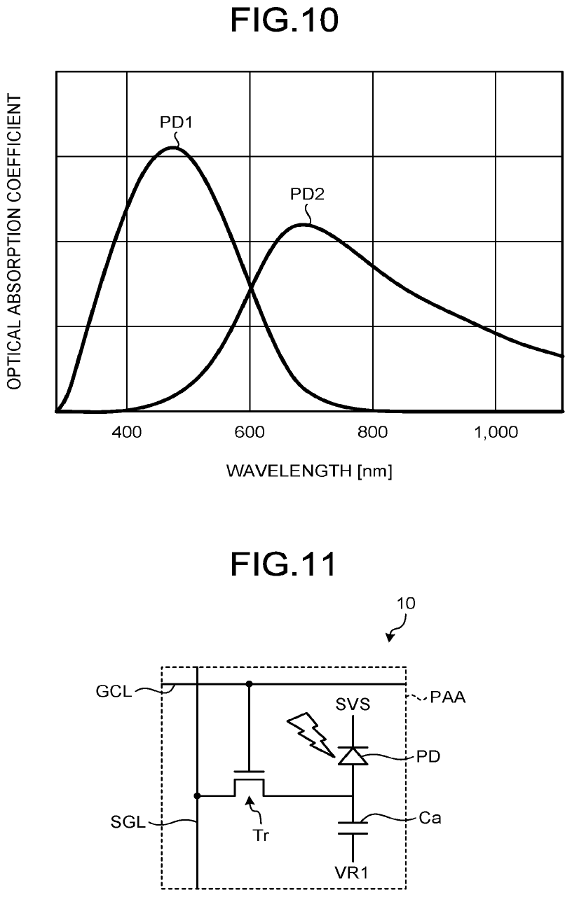

FIG. 10 is a graph schematically illustrating a relation between a wavelength and an optical absorption coefficient of each of the first photodiode and the second photodiode. In FIG. 10, the horizontal axis represents the wavelength, and the vertical axis represents the optical absorption coefficient. The optical absorption coefficient is an optical constant that represents a degree of absorption of light traveling through a substance.

As illustrated in FIG. 10, the first photodiode PD1 containing a-Si exhibits a good optical absorption coefficient in the visible light range, for example, in a wavelength range from 300 nm to 800 nm. In contrast, the second photodiode PD2 containing polysilicon exhibits a good optical absorption coefficient in a range of, for example, from 500 nm to 1100 nm, including visible to infrared ranges. In other words, the first photodiode PD1 has high sensitivity in the visible light range, and the second photodiode PD2 has high sensitivity in a range from the red wavelength range to the infrared range that differs from the range of the first photodiode PD1.

In the portable terminal 100 of the present embodiment, the first and the second photodiodes PD1 and PD2 having different sensitive wavelength ranges are stacked. With this configuration, the wavelength range having high sensitivity can be wider than in a configuration including only either of the photodiodes.

The light L1 (refer to FIG. 3) penetrates the portable terminal 100 through the space SP and the space SPa. The light L2 reflected by the finger Fg (refer to FIG. 3) enters the first photodiode PD1. Of the light L2, light in a wavelength range not absorbed by the first photodiode PD1 passes through the first photodiode PD1, and enters the second photodiode PD2. For example, in the fingerprint detection, the first photodiode PD1 can well detect the blue or green light L2. In the vascular pattern (for example, vein pattern) detection, the infrared light L2 is not absorbed by the first photodiode PD1, and enters the second photodiode PD2. Thus, the second photodiode PD2 can well detect the infrared light L2. As a result, the portable terminal 100 can detect the various types of biological information using the same device (portable terminal 100).

Even if the i region 52a of the second photodiode PD2 has changed to the n-type under the influence of electrical charges or impurities of the insulating films including, for example, the first interlayer insulating film 24, the i region 52a is neutralized by the cathode electrode 34 of the first photodiode PD1. As a result, the portable terminal 100 can be increased in optical sensitivity.

The first and the second photodiodes PD1 and PD2 are provided in the partial detection area PAA, that is, in the area surrounded by the gate lines GCL and the signal lines SGL. With this configuration, the number of switching elements and the number of wires can be smaller than in a case where each of the first and the second photodiodes PD1 and PD2 is provided with the first switching element Tr, the gate line GCL, and the signal line SGL. Accordingly, the portable terminal 100 can improve the resolution of the detection.

As described above, the sensor 10 includes the first photodiode PD1 including the first semiconductor layer 31 containing amorphous silicon and the second photodiode PD2 including the second semiconductor layer 51 containing polysilicon. In the sensor 10, the first semiconductor layer 31 containing amorphous silicon and the second semiconductor layer 51 containing polysilicon, that is, the first and the second photodiodes PD1 and PD2 are stacked so as to overlap each other in the third direction Dz. However, in the sensor 10, the first and the second photodiodes PD1 and PD2 need not be stacked in the third direction Dz, and may be provided, for example, in the same layer.

The sensor 10 can detect, as the biological information, the fingerprint of the user using the first photodiode PD1, and the vascular pattern of the user using the second photodiode PD2. The vascular pattern refers to an image of blood vessels, and is the vein pattern in the present embodiment. Although the sensor 10 detects the fingerprint and the vascular pattern as the biological information on the user, the sensor 10 may detect at least one of the fingerprint and the vascular pattern. The sensor 10 may detect the biological information (for example, pulsation and/or a pulse wave) other than the fingerprint and the vascular pattern.

An exemplary case will be described where the sensor 10 detects only one of the fingerprint and the vascular pattern. The following describes an exemplary case where the sensor 10 detects the vascular pattern without detecting the fingerprint. FIG. 11 is an equivalent circuit diagram illustrating the partial detection area according to another example. As illustrated in FIG. 11, the sensor 10 in this example has the partial detection areas PAA arranged in a matrix having a row-column configuration. As illustrated in FIG. 11, the partial detection areas PAA of the sensor 10 include the second photodiode PD2, the capacitive element Ca, and the first switching element Tr. The first switching element Tr is provided correspondingly to the second photodiode PD2. The gate of the first switching element Tr is coupled to the gate line GCL. The source of the first switching element Tr is coupled to the signal line SGL. The drain of the first switching element Tr is coupled to the cathode electrode 54 of the second photodiode PD2 and one end of the capacitive element Ca. The anode electrode 55 of the second photodiode PD2 and the other end of the capacitive element Ca are coupled to the reference potential, for example, the ground potential. That is, the sensor 10 has a configuration not including the first photodiode PD1.

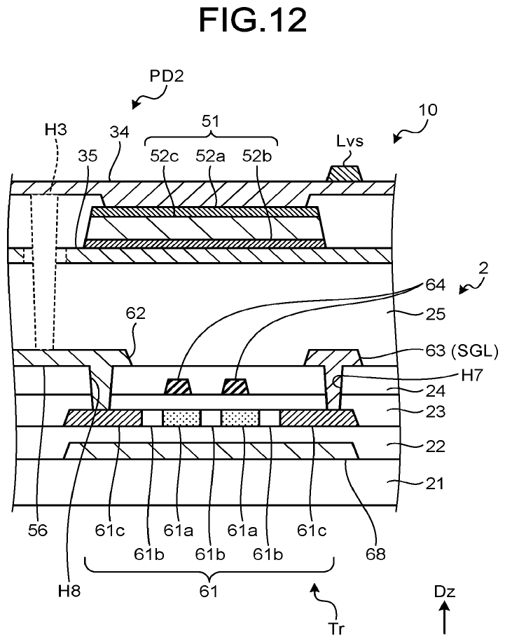

FIG. 12 is a schematic sectional view of the partial detection area according to the other example. As illustrated in FIG. 12, the sensor 10 in this example is provided with the first switching element Tr above the insulating substrate 21 in the same manner as in FIG. 9. However, unlike in FIG. 9, the sensor 10 in this example is not provided with the first photodiode PD1. In addition, the sensor 10 in this example is provided with the second photodiode PD2 at a location different from that in FIG. 9. In the sensor 10 of this example, the second photodiode PD2 is provided on the upper side, that is, in the third direction Dz of the first switching element Tr. That is, the anode electrode 35 of the second photodiode PD2 is provided above the second interlayer insulating film 25. The second photodiode PD2 is stacked in the order of the anode electrode 35, the second semiconductor layer 51, and the cathode electrode 34. The second semiconductor layer 51 is stacked in the order of the p region 52b, the i region 52a, and the n region 52c above the anode electrode 35. The anode electrode 35 is coupled to the source electrode 62 of the first switching element Tr through the contact hole H4 provided in the second interlayer insulating film 25.

As described above, the sensor 10 includes the second photodiode PD2 including the second semiconductor layer 51 containing polysilicon, and need not include the first photodiode PD1. In this case, the sensor 10 includes the second photodiode PD2, and thus, can suitably detect the vascular pattern of the user.

When the sensor 10 is a sensor that detects the fingerprint of the user and does not detect the vascular pattern of the user, the sensor 10 has a configuration including the first photodiode PD1 without including the second photodiode PD2. In that case, the equivalent circuit of the sensor 10 is preferably obtained by replacing the second photodiode PD2 in FIG. 11 with the first photodiode PD1, and the stacking configuration of the sensor 10 is preferably obtained by replacing the second photodiode PD2 in FIG. 12 with the first photodiode PD1.

While the stacking configuration of the sensor 10 has been described above, the structure of the sensor 10 is not limited to that described above, and may be any structure as long as being capable of detecting the biological information on the user.

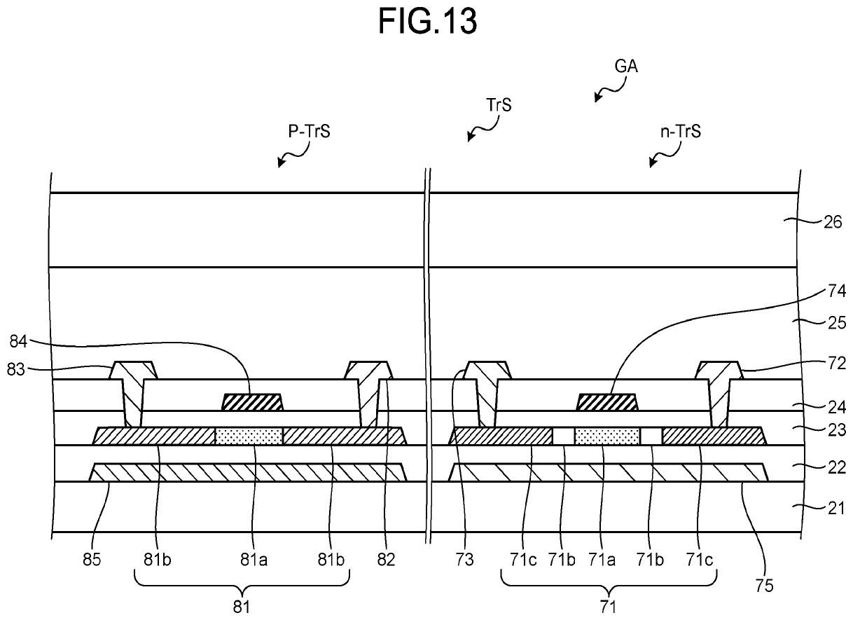

The following describes a stacking configuration of the third switching element TrS. FIG. 13 is a sectional view illustrating a schematic sectional configuration of the switching element included in the drive circuit. FIG. 13 explains the third switching element TrS included as a drive circuit switching element in the signal line selection circuit 16. However, the explanation of FIG. 13 can also be applied to switching elements included in other drive circuits. That is, the same configuration as that of FIG. 13 can be applied to the second switching elements TrG included in the gate line drive circuit 15 and the fourth switching element TrR included in the reset circuit 17.

As illustrated in FIG. 13, the n-channel transistor n-TrS of the third switching element TrS includes a fourth semiconductor layer 71, a source electrode 72, a drain electrode 73, and a gate electrode 74. The p-channel transistor p-TrS includes a fifth semiconductor layer 81, a source electrode 82, a drain electrode 83, and a gate electrode 84. A light-blocking layer 75 is provided between the fourth semiconductor layer 71 and the insulating substrate 21. A light-blocking layer 85 is provided between the fifth semiconductor layer 81 and the insulating substrate 21.

Both the fourth semiconductor layer 71 and the fifth semiconductor layer 81 are of polysilicon. The fourth semiconductor layer 71 and the fifth semiconductor layer 81 are more preferably of LTPS. The fourth semiconductor layer 71 includes an i region 71a, LDD regions 71b, and the n regions 61c. The fifth semiconductor layer 81 includes an i region 81a and p regions 81b.

The n-channel transistor n-TrS and the p-channel transistor p-TrS have the same layer configuration as that of the first switching element Tr illustrated in FIG. 9. That is, the fourth semiconductor layer 71 and the fifth semiconductor layer 81 are provided in the same layer as those of the second semiconductor layer 51 and the third semiconductor layer 61 illustrated in FIG. 9; the gate electrode 74 and the gate electrode 84 are provided in the same layer as those of the gate electrodes 64 illustrated in FIG. 9; and the source electrode 72, the drain electrode 73, the source electrode 82, and the drain electrode 83 are provided in the same layer as those of the source electrode (first relay electrode 56) and the drain electrode 63 (signal line SGL) illustrated in FIG. 9.

As described above, the first photodiode PD1 and the first switching element Tr provided in the detection area AA use the same material and are provided in the same layer as the switching elements are, such as the third switching element TrS provided in the peripheral area GA. This configuration can simplify the manufacturing process and reduce the manufacturing cost of the portable terminal 100. The drive circuit provided in the peripheral area GA is not limited to being constituted by the CMOS transistor, and may be constituted by either the n-channel transistor n-TrS or the p-channel transistor p-TrS.

Function Execution Device

The portable terminal 100 has the above-described configuration. The following describes a configuration of the function execution device 110.

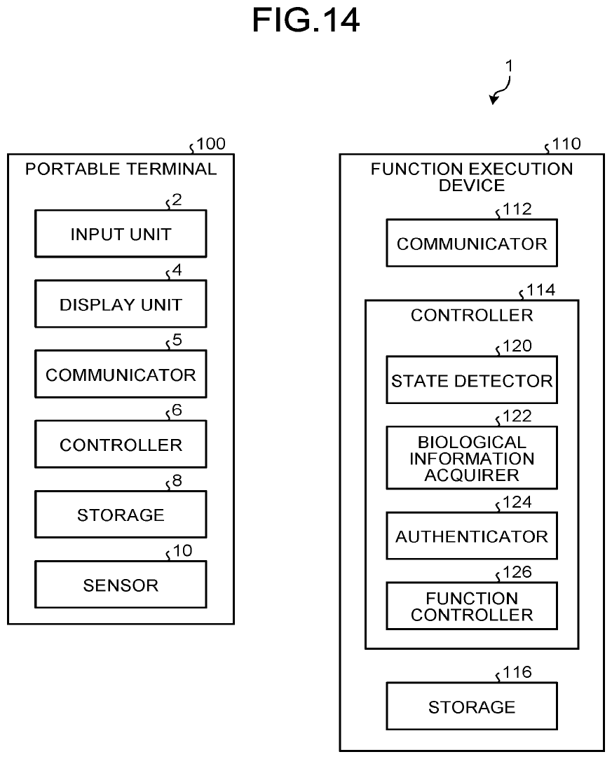

FIG. 14 is a block diagram illustrating functional configurations of the function execution device and the portable terminal according to the first embodiment. As illustrated in FIG. 14, the portable terminal 100 includes an input unit 2, a display unit 4, and a communicator 5, and also includes the controller 6, the storage 8, and the sensor 10 described above. The input unit 2 is an input device for receiving the operations of the user U. The display unit 4 is a display for displaying the image. In the first embodiment, the input unit 2 and the display unit 4 overlap each other to constitute a touchscreen panel. The communicator 5 is configured to be controlled by the controller 6 to communicate with external equipment such as the function execution device 110. That is, the communicator 5 is a communication interface for performing the communication. The portable terminal 100 wirelessly communicates with the function execution device 110. For example, Wi-Fi or Bluetooth (registered trademark) is used as a method for the wireless communication.

As described above, the controller 6 acquires the biological information on the user U detected by the sensor 10. The controller 6 transmits the biological information on the user U through the communicator 5 to the function execution device 110.

As illustrated in FIG. 14, the function execution device 110 includes a communicator 112, a controller 114, and a storage 116. The communicator 112 is configured to be controlled by the controller 114 to communicate with external equipment such as the portable terminal 100. That is, the communicator 112 is a communication interface for performing the communication. The controller 114 is an arithmetic device, that is, a CPU mounted on the function execution device 110. The controller 114 performs various types of processing, for example, by reading a computer program from the storage 116. The storage 116 is a memory for storing therein, for example, content of arithmetic operations and information on the computer program of the controller 114, and includes at least one of external storage devices such as a RAM, a ROM, and an HDD.

The controller 114 includes a state detector 120, a biological information acquirer 122, an authenticator 124, and a function controller 126. The controller 114 reads software (computer program) from the storage 116 to implement the state detector 120, the biological information acquirer 122, the authenticator 124, and the function controller 126, and executes the processing described below.

The state detector 120 detects whether the portable terminal 100 is in a predetermined state. The term “predetermined state” refers to a state where the portable terminal 100 is in a state set in advance. In the first embodiment, the predetermined state refers to that the portable terminal 100 and the function execution device 110 are within a predetermined distance from each other. For example, the portable terminal 100 acquires position information on the portable terminal 100 at predetermined intervals of time. The portable terminal 100 may acquire the position information on the portable terminal 100, for example, via the Global Positioning System (GPS). The portable terminal 100 causes the sensor 10 to detect the biological information on the user U at the time when the position information on the portable terminal 100 is acquired. In other words, the portable terminal 100 acquires the position information on the portable terminal 100 at the time when the sensor 10 detects the biological information on the user U, that is, at the time when the finger Fg or the palm is in proximity to a place where the sensor 10 is located. The state detector 120 acquires the position information on the portable terminal 100 from the portable terminal 100 through the communicator 112. The state detector 120 calculates the distance between the function execution device 110 and the portable terminal 100 based on the acquired position information on the portable terminal 100, and determines whether the distance between the function execution device 110 and the portable terminal 100 is within the predetermined distance set in advance. If so, the state detector 120 determines that the portable terminal 100 is in the predetermined state, or if not, the state detector 120 determines that the portable terminal 100 is not in the predetermined state. The state detector 120 may acquire position information on the function execution device 110 by reading the position information on the function execution device 110 stored in advance from the storage 116, and calculate the distance between the function execution device 110 and the portable terminal 100 based on the position information on the function execution device 110 and the position information on the portable terminal 100. However, any method may be used to calculate the distance between the function execution device 110 and the portable terminal 100. For example, the function execution device 110 may detect that the portable terminal 100 is in proximity to the function execution device 110, by receiving a signal emitted by the portable terminal 100 via Wi-Fi or Bluetooth (registered trademark). The detection system 1 may have a function to inform the user U that the portable terminal 100 is in proximity to the function execution device 110, for example, via the screen of the portable terminal or a vibration function of the portable terminal.

The predetermined state detected by the state detector 120 is not limited to the state where the portable terminal 100 and the function execution device 110 are within the predetermined distance from each other, and may be any state set in advance. For example, the state detector 120 may set the predetermined state to a state where the predetermined function is requested to be executed. In this case, for example, the user U enters an operation requesting the function execution device 110 to execute the predetermined function to the portable terminal 100. Alternatively, the operation may be requested to the user U, for example, via the screen of the portable terminal 100 or the vibration function of the portable terminal 100 that informs the user U that the portable terminal 100 is in proximity to the function execution device 110, as described above. When the input unit 2 receives the operation of the user U requesting the execution of the predetermined function, the portable terminal 100 generates a signal requesting the execution of the predetermined function. The portable terminal 100 causes the sensor 10 to detect the biological information on the user U at the time when the signal requesting the execution of the predetermined function is generated. The state detector 120 acquires the signal requesting the execution of the predetermined function generated by the portable terminal 100. In this case, the state detector 120 determines that the portable terminal 100 is in the predetermined state when the signal requesting the execution of the predetermined function is acquired. The user U may enter the operation requesting the function execution device 110 to execute the predetermined function to the function execution device 110. In this case, the state detector 120 determines that the portable terminal 100 is in the predetermined state when the state detector 120 receives the operation requesting the execution of the predetermined function. When the user U operates the function execution device 110, the portable terminal 100 carried by the user U can be said to be in a position close to the function execution device 110. Accordingly, the fact that the user U operates the function execution device 110 may be said that the portable terminal 100 is in the predetermined state. The state detector 120 may determine that the portable terminal 100 is in the predetermined state when the predetermined function is requested to be executed and the portable terminal 100 and the function execution device 110 are within the predetermined distance from each other. The state detector 120 is provided in the function execution device 110, but may be provided in the portable terminal 100.

The biological information acquirer 122 acquires the biological information on the user U detected by the sensor 10 from the portable terminal 100 through the communication. The biological information acquirer 122 acquires the biological information on the user U detected by the sensor 10 from the portable terminal 100, when triggered because the state detector 120 has determined that the portable terminal 100 is in the predetermined state; and in this case, the biological information acquirer 122 is triggered because the state detector 120 has determined that the portable terminal 100 and the function execution device 110 are within the predetermined distance from each other. More specifically, the biological information acquirer 122 preferably acquires the biological information on the user U detected by the sensor 10 when the portable terminal 100 is determined to be in the predetermined state. That is, for example, in the first embodiment, the biological information acquirer 122 preferably acquires the biological information on the user U detected by the sensor 10 at the time when the position information on the portable terminal 100 used when the portable terminal 100 is determined to be in the predetermined state is acquired. Through this operation, the biological information acquirer 122 can acquire the biological information on the user U at the time when the portable terminal 100 is in the predetermined state, in this case, at the time when the portable terminal 100 and the function execution device 110 are within the predetermined distance from each other.

The authenticator 124 performs the authentication of the user U based on the biological information on the user U acquired by the biological information acquirer 122, and determines whether to execute the predetermined function. As describe above, the predetermined function refers to a function (for example, unlocking) set in advance to be executed by the function execution device 110. The authenticator 124 reads, from the storage 116, reference biological information that is the biological information serving as a reference stored in advance. The reference biological information is stored in advance as, for example, the biological information on the user (herein, the two-dimensional information on the fingerprint or the vascular pattern) based on which the user is allowed to use the predetermined function. The reference biological information is not limited to being stored in the storage 116, and may be acquired, for example, from an external device through communication. The authenticator 124 performs the authentication by checking for a match between the biological information on the user U and the reference biological information to determine whether the biological information on the user U matches with the reference biological information. For example, the authenticator 124 may check for a pattern match between the biological information on the user U and the reference biological information, and may determine that the biological information on the user U matches with the reference biological information if the degree of similarity of feature points is equal to or higher than a predetermined degree, or determine that the biological information on the user U does not match with the reference biological information if the degree of similarity is lower than the predetermined degree. A known technique may be used to check for a match between the biological information on the user U and the reference biological information.

If the authenticator 124 determines that the biological information on the user U matches with the reference biological information, the authenticator 124 determines that the user U has been authenticated, and determines to execute the predetermined function. If, instead, the authenticator 124 determines that the biological information on the user U does not match with the reference biological information, the authenticator 124 determines that the user U cannot be authenticated, and determines not to execute the predetermined function.

The function controller 126 controls the function execution device 110 to cause the function execution device 110 to execute the predetermined function. The function controller 126 causes the function execution device 110 to execute the predetermined function if the authenticator 124 determines to execute the predetermined function, that is, determines that the user has been authenticated. The function controller 126 does not cause the function execution device 110 to execute the predetermined function if the authenticator 124 determines not to execute the predetermined function, that is, determines that the user cannot be authenticated.

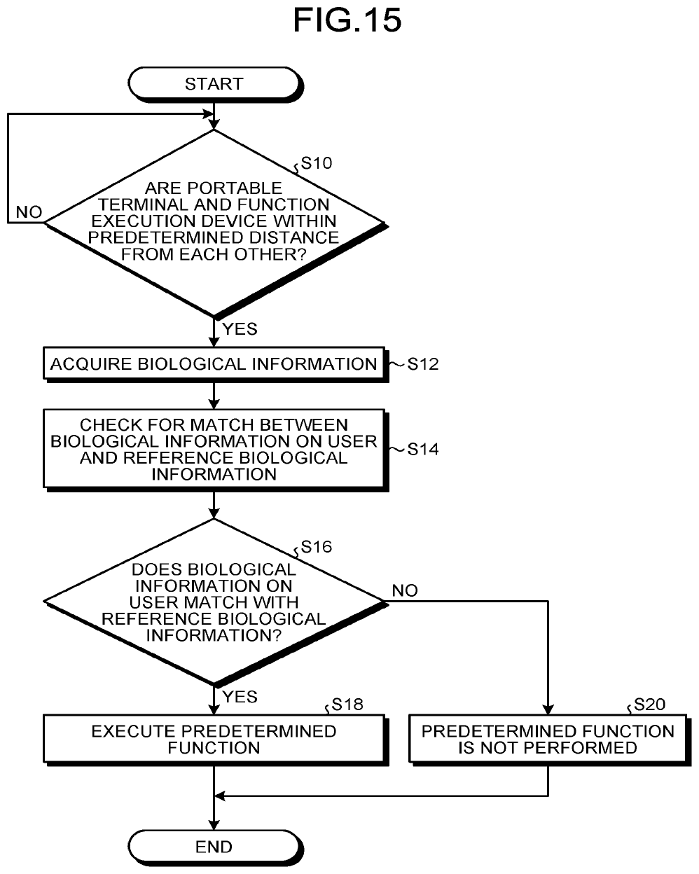

The function execution device 110 has the above-described configuration. The following describes a flow of authentication processing performed by the function execution device 110 based on a flowchart. FIG. 15 is the flowchart explaining the authentication processing according to the first embodiment. As illustrated in FIG. 15, the function execution device 110 causes the state detector 120 to determine whether the portable terminal 100 is in the predetermined state, in this case, whether the portable terminal 100 and the function execution device 110 are within the predetermined distance from each other (Step S10), and if so (Yes at Step S10), causes the biological information acquirer 122 to acquire the biological information on the user U detected by the sensor 10 (Step S12). The biological information acquirer 122 acquires the biological information on the user U detected by the sensor 10 when the position information on the portable terminal 100 is acquired. If the portable terminal 100 and the function execution device 110 are not within the predetermined distance from each other (No at Step S10), that is, if the portable terminal 100 is not in the predetermined state, the process returns to Step S10.

After the biological information is acquired, the function execution device 110 causes the authenticator 124 to check for a match between the acquired biological information on the user U and the reference biological information to perform the authentication of the user U (Step S14). If the biological information on the user U matches with the reference biological information (Yes at Step S16), the function execution device 110 causes the authenticator 124 to determine to execute the predetermined function, and causes the function controller 126 to execute the predetermined function (in this case, unlocking) (Step S18). If the biological information on the user U does not match with the reference biological information (No at Step S16), the function execution device 110 causes the authenticator 124 to determine not to execute the predetermined function, and causes the function controller 126 not to execute the predetermined function (Step S20), that is, in this case, not to perform the unlocking. This process ends after Step S18 or Step S22. However, even if the user is determined to be not authenticated and the processing at Step S20 is performed, the process may return to Step S10 or Step S12 to continue again the authentication processing.

In the first embodiment, the function execution device 110 includes the authenticator 124 to perform the authentication. However, the function execution device 110 need not perform the authentication. In this case, the function execution device 110 may transmit the acquired biological information on the user U to another server that includes the authenticator 124, and this server may perform the authentication. The function execution device 110 then acquires the result of the authentication by the server, that is, the result of the determination on whether to execute the predetermined function, and causes the function controller 126 to execute the predetermined function based on the result of the determination.

As described above, the detection system 1 according to the present embodiment includes the portable terminal 100 serving as a portable object and the function execution device 110. The portable terminal 100 is a terminal that includes the sensor 10 for detecting the biological information on the user U and is portable by the user U. The function execution device 110 causes the biological information acquirer 122 to acquire, through the communication, the biological information on the user U detected by the sensor 10. The function execution device 110 causes the function controller 126 to execute the predetermined function based on the authentication result of the user U associated with the biological information on the user U. In the detection system 1, the portable terminal 100 carried by the user U detects the biological information on the user U. The function execution device 110 acquires, through the communication, the biological information on the user U detected by the sensor 10. If the user U has been authenticated in the authentication result based on the biological information, the function execution device 110 executes the predetermined function. Accordingly, since the detection system 1 allows the biological information to be detected by the portable terminal 100 carried by the user U, the user U need not operate the function execution device 110 in order to be authenticated. In addition, the user U carries the portable terminal 100. Therefore, when the portable terminal 100 detects the biological information, the biological information can be detected while the user U simply holds the portable terminal 100. Thus, the user U need not perform an unaccustomed operation for the authentication. Thus, the detection system 1 can reduce time and effort for the authentication.

The function execution device 110 acquires the biological information on the user U from the sensor 10, when triggered because the portable terminal 100 has been brought into the predetermined state. At this time, the function execution device 110 preferably executes the predetermined function at an appropriate time for the user U. In that respect, the function execution device 110 of the present embodiment acquires the biological information on the user U and executes the predetermined function based on the authentication result, when triggered because the portable terminal 100 has been brought into the predetermined state. Accordingly, the detection system 1 can execute the predetermined function at the time when the portable terminal 100 is brought into the predetermined state. As a result, the predetermined function can be executed at the appropriate time for the user U.

The function execution device 110 acquires the biological information on the user U from the sensor 10 and executes the predetermined function based on the authentication result, when triggered because the portable terminal 100 and the function execution device 110 are within the predetermined distance from each other. Accordingly, the detection system 1 can execute the predetermined function at the time when the user is close to the function execution device 110. As a result, the predetermined function can be executed at the appropriate time for the user U. For example, in a case where the function execution device 110 performs the unlocking, even if the unlocking is performed when the user is in a distant position, the locking may be performed or someone else may enter the room before the user U reaches the room. In contrast, the detection system 1 can execute the predetermined function at the time when the user is close to the function execution device 110. As a result, such a problem can be restrained from occurring. In addition, with the detection system 1 of the present embodiment, each user need not be individually subjected to the personal authentication, for example, via a sensor provided at the function execution device 110 when the user is going to enter the room. Thus, the user can, for example, smoothly enter the room. However, since no gate is required to let each user individually pass therethrough, even an unauthorized person may enter the room. Therefore, a mechanism may be provided that upon the biological information acquired by the portable terminal 100 being transferred to the function execution device 110 when the user passes through the gate that can let each user individually pass therethrough, the authentication is performed by checking the biological information for a match with the reference biological information when the user is passing through the gate, and the gate is closed before the user completes passing through the gate if the authentication has failed. The detection system 1 may have a configuration in which the above-described gate is provided with a sensor, and the sensor provided at the gate is used to authenticate the user not carrying the portable terminal 100.

The sensor 10 detects at least one of the vascular pattern of the user U and the fingerprint of the user U. This detection system 1 can appropriately perform the authentication of the user U by detecting the vascular pattern and/or the fingerprint as the biological information.

The sensor 10 includes the semiconductor (first semiconductor layer 31) containing amorphous silicon and the semiconductor (second semiconductor layer 51) containing polysilicon, and detects the vascular pattern of the user and the fingerprint of the user. By including such a sensor 10, the detection system 1 can perform the authentication based on a plurality of types of the biological information, and thus, can increase the accuracy of the authentication. For example, the detection system 1 may determine that the user has been authenticated and execute the predetermined function if both the fingerprint and the vascular pattern of the user match with those of the reference biological information. Alternatively, the detection system 1 may acquire one of the fingerprint and the vascular pattern of the user, and, if the acquired one matches with the reference biological information, may determine that the user has been authenticated and execute the predetermined function. The detection system 1 may then acquire the other of the fingerprint and the vascular pattern of the user, and may halt the execution of the predetermined function if the other does not match with the reference biological information.

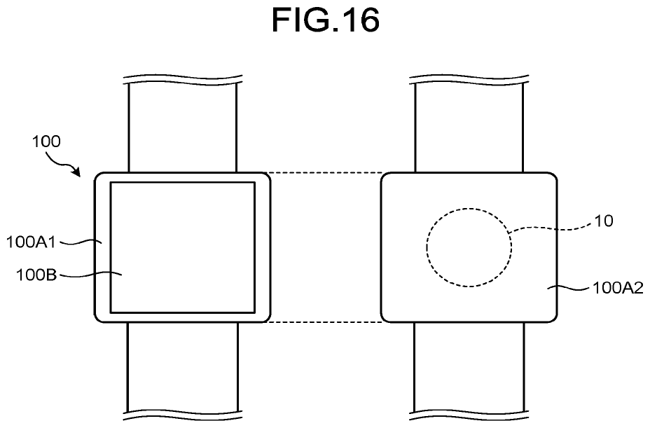

The portable terminal 100 according to the present embodiment is a smartphone or a tablet computer held and operated by the user, but is not limited thereto, and may be any terminal. FIG. 16 is a diagram illustrating another example of the portable terminal. For example, as illustrated in FIG. 16, the portable terminal 100 may be a wristwatch including the sensor 10, that is, what is called a smart watch. In this case, the sensor 10 is preferably provided on the back face 100A2 that is a face on the opposite side of the front face 100A1 provided with the display area 100B for displaying, for example, time. In this case, since the back face 100A2 side is always in contact with an arm of the user U, the sensor 10 can easily detect the biological information, and thereby, the time and effort for the authentication can be reduced.

### Second Embodiment

The following describes a second embodiment of the present disclosure. In the second embodiment, a function execution device 110a differs from the function execution device 110 of the first embodiment. While the function execution device 110 of the first embodiment operates the device to be operated 200 serving as another device as the predetermined function, the function execution device 110a of the second embodiment operates the function execution device 110a itself as the predetermined function. In the second embodiment, portions having configurations common to those of the first embodiment will not be described.

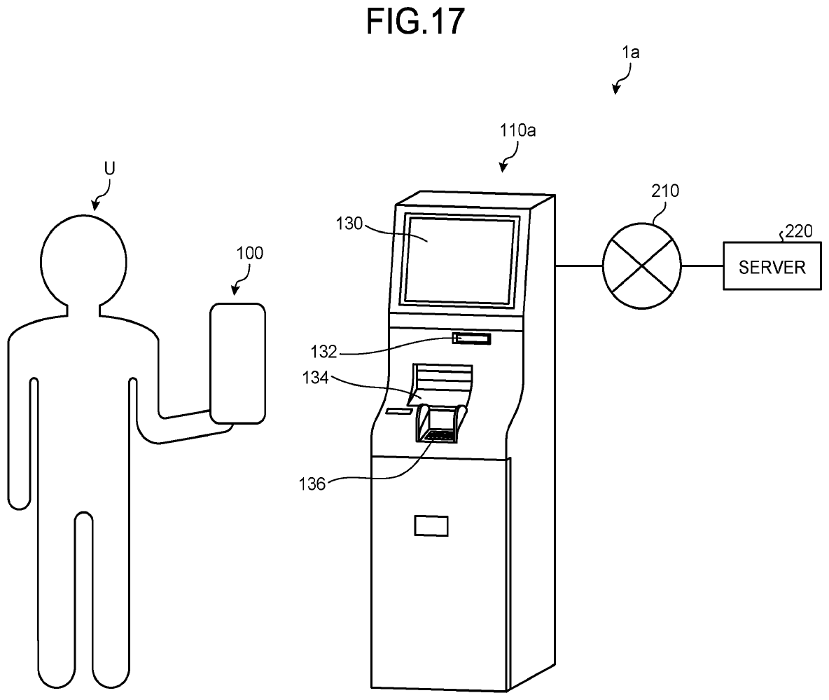

FIG. 17 is a schematic diagram of a detection system according to the second embodiment. As illustrated in FIG. 17, a detection system 1a according to the second embodiment includes the portable terminal 100 and the function execution device 110a. In the example of FIG. 17, the function execution device 110a is a device that allows deposit and withdrawal of cash, and is, for example, an automated teller machine (ATM) installed in, for example, a financial facility or a multifunction device that handles cash and is installed in, for example, a convenience store. The function execution device 110a includes a display unit 130, a card insertion unit 132, an input unit 134, and a banknote processor 136.

The display unit 130 is a screen for displaying, for example, operation details. The display unit 130 may be a touchscreen panel provided with an overlapping input unit for receiving operations of the user U. The card insertion unit 132 is configured to allow insertion and discharge of a card in a transaction using the card, such as a cash card. The card insertion unit 132 discharges a receipt issued when the transaction is finished. The input unit 134 is a device for receiving the operations of the user U, and is, for example, a keyboard. The banknote processor 136 receives and delivers banknotes at the times of deposit and withdrawal transactions.

In the same manner as the function execution device 110 of the first embodiment, the function execution device 110a includes the communicator 112, the controller 114, and the storage 116. However, the function execution device 110a does not include the authenticator 124 for performing the authentication processing. The function execution device 110a is coupled to a server 220 serving as external equipment through a network 210, and transmits and receives information to and from the server 220. The server 220 includes a controller serving as a CPU and a storage serving as a memory. The controller includes the authenticator 124 for performing the authentication processing.

The function execution device 110a acquires the biological information on the user U from the portable terminal 100 and executes the predetermined function, when triggered because the user U has entered an execution request of the predetermined function to the function execution device 110a. That is, the user U operates the input unit 134 or the input unit overlapping the display unit 130 of the function execution device 110a to enter an operation to request the function execution device 110a to execute the predetermined function. In this case, the predetermined function is, for example, the deposit or the withdrawal of cash. The function execution device 110a acquires the biological information on the user U from the portable terminal 100 and executes the predetermined function, when triggered because the input unit has received the execution request of the predetermined function from the user U. That is, the function execution device 110a detects, as the predetermined state, that the execution request of the predetermined function is issued to the function execution device 110a.

The function execution device 110a may acquire the biological information on the user U from the portable terminal 100 and execute the predetermined function, when triggered because the execution request of the predetermined function to the function execution device 110a has been entered to the portable terminal 100. In this case, the user U enters the request to the function execution device 110a to execute the predetermined function to the portable terminal 100. In this case, the portable terminal 100 generates a signal for requesting the execution of the predetermined function, and transmits the signal to the function execution device 110a. When the signal requesting the execution of the predetermined function is acquired, the function execution device 110a determines that the portable terminal 100 is in the predetermined state, and acquires the biological information on the user U from the portable terminal 100, and executes the predetermined function.

That is, the function execution device 110a may acquire the biological information on the user U from the sensor 10 and execute the predetermined function based on the authentication result, when triggered because the user U has performed the operation for executing the predetermined function to the portable terminal 100 or the function execution device 110a.

The function execution device 110a transmits the acquired biological information on the user U to the server 220 through the network 210. The server 220 uses the same method as that of the authenticator 124 of the first embodiment to perform the authentication based on the biological information on the user U. The server 220 transmits the result of the authentication, that is, the result of the determination on whether to execute the predetermined function, to the function execution device 110a. The function execution device 110a acquires the authentication result by the server 220, that is, the result of the determination on whether to execute the predetermined function, and causes the function controller 126 to execute the predetermined function based on the result of the determination. However, the function execution device 110a may include the authenticator 124 and perform the authentication by itself in the same manner as in the first embodiment, without communicating with the server 220.

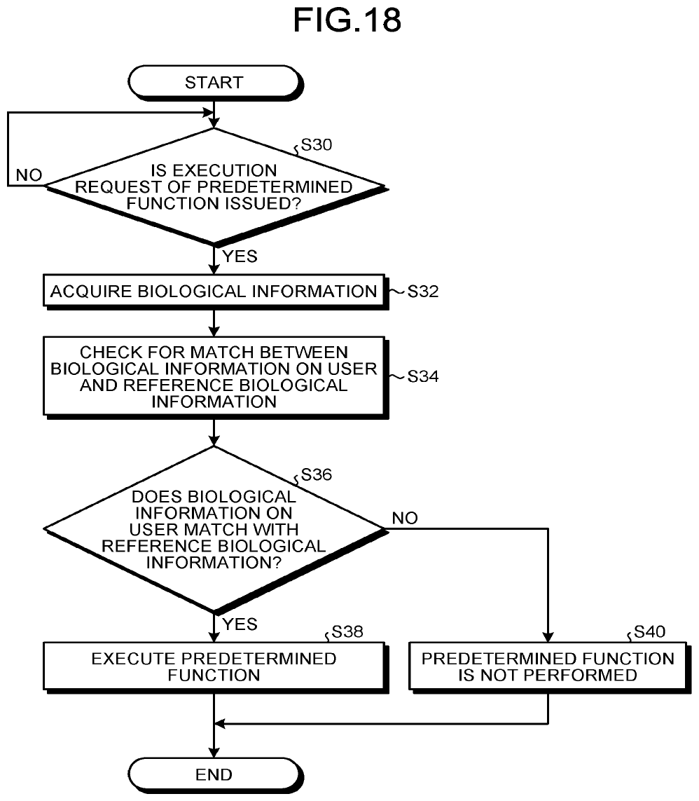

FIG. 18 is a flowchart explaining the authentication processing according to the second embodiment. As illustrated in FIG. 18, the function execution device 110a causes the state detector 120 to determine whether the portable terminal 100 is in the predetermined state, in this case, whether the operation to request the function execution device 110a to execute the predetermined function is performed (Step S30), and if the execution request of the predetermined function is issued (Yes at Step S30), the function execution device 110a causes the biological information acquirer 122 to acquire the biological information on the user U detected by the sensor 10 (Step S32). The biological information acquirer 122 acquires the biological information on the user U detected by the sensor 10 when the portable terminal 100 is determined to be in the predetermined state, in other words, when the operation to request the function execution device 110a to execute the predetermined function is performed. If the execution request of the predetermined function is not issued (No at Step S30), that is, if the portable terminal 100 is not in the predetermined state, the process returns to Step S30. The processing at and after Step S32 is the same as that of the first embodiment, that is, the processing at and after Step S12 in FIG. 15, and therefore will not be described.

As described above, the function execution device 110a acquires the biological information on the user U from the sensor 10 and executes the predetermined function based on the authentication result, when triggered because the user U has performed the operation for executing the predetermined function to the portable terminal 100 or the function execution device 110a. Accordingly, the detection system 1 according to the present embodiment can execute the predetermined function at the time at which the predetermined function is required for the user U to be performed. As a result, the predetermined function can be executed at the appropriate time for the user U. Since the user need not perform an operation for the authentication to the function execution device 110a, the time and effort for the authentication can be reduced. The function execution device 110a may acquire the biological information on the user U from the sensor 10 and execute the predetermined function based on the authentication result if the portable terminal 100 and the function execution device 110 are within the predetermined distance from each other and also the user U performs the operation for executing the predetermined function to the portable terminal 100 or the function execution device 110a. In this case, the predetermined function can be executed at a further appropriate time for the user U.

### Third Embodiment

The following describes a third embodiment of the present disclosure. The third embodiment differs from the first embodiment in that the portable object provided with the sensor 10 is a card 100b. In the third embodiment, portions having configurations common to those of the first embodiment will not be described.

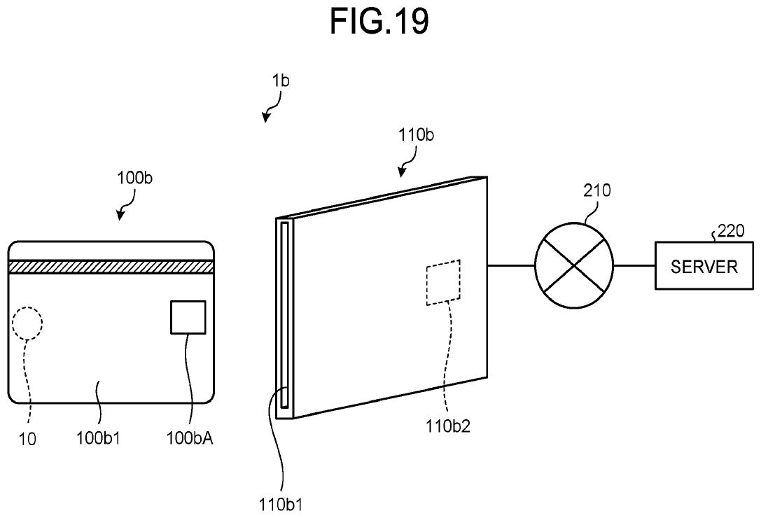

FIG. 19 is a schematic diagram of a detection system according to the third embodiment. As illustrated in FIG. 19, a detection system 1b according to the third embodiment includes the card 100b serving as the portable object and a function execution device 110b. The card 100b is a card portable by the user U, and includes a storage 100bA and the sensor 10 on a back face 100b1. The storage 100bA is a terminal, in this case, an integrated circuit (IC) chip that stores therein information on the card 100b. The storage 100bA stores therein, for example, identification (ID) information on the card 100b. The sensor 10 and the storage 100bA are provided on the back face 100b1, that is, the same face, but may be provided on different faces. The card 100b is, for example, a credit card.

The function execution device 110b is a device that reads the ID information from the card 100b, and executes the predetermined function. The function execution device 110b, for example, makes payment from the card 100b as the predetermined function. The function execution device 110b includes an insertion portion 110b1 that is a slot through which the card 100b is insertable and a reading unit 110b2 serving as a terminal for reading the information, such as the ID information, stored in the card from the storage 100bA of the card 100b. The function execution device 110b also includes the communicator 112, the controller 114, and the storage 116 in the same manner as the function execution device 110 of the first embodiment. However, the function execution device 110a does not include the authenticator 124 for performing the authentication processing. The function execution device 110a is coupled to the server 220 serving as the external equipment through the network 210, and transmits and receives information to and from the server 220. The server 220 includes the controller serving as the CPU and the storage serving as the memory. The controller includes the authenticator 124 for performing the authentication processing.

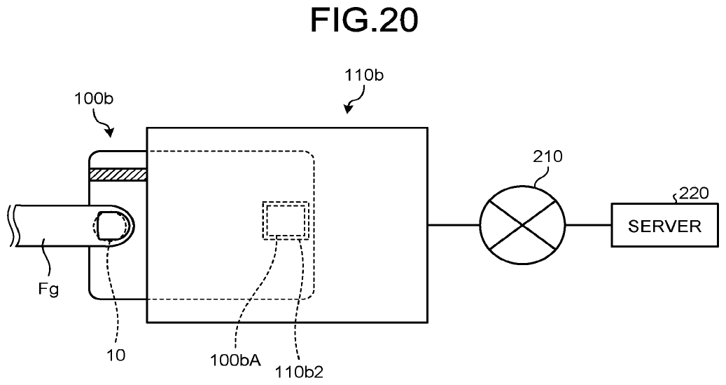

FIG. 20 is a schematic diagram illustrating an exemplary state where the card is inserted in the function execution device. As illustrated in FIG. 20, when the function execution device 110b is caused to execute the predetermined function, the card 100b is inserted from the insertion portion 110b1 into the function execution device 110b. The card 100b is inserted into the function execution device 110b such that the storage 100bA faces the reading unit 110b2 of the function execution device 110b. The user U inserts the card 100b into the function execution device 110b in a state of holding the sensor 10 of the card 100b (in the state where the finger Fg of the user is in proximity to the sensor 10).

In this state, the function execution device 110b reads the ID information stored in the storage 100bA from the reading unit 110b2. In the state where the card 100b is inserted in the function execution device 110b, the function execution device 110b supplies power, for example, to the sensor 10 of the card 100b to drive the sensor 10. The driving of the sensor 10 causes the card 100b to detect the biological information on the user U, and the detected biological information is transmitted to the function execution device 110b. That is, the function execution device 110b acquires the biological information on the user U from the card 100b, when triggered because the card 100b has been inserted into the function execution device 110b, and more specifically, the function execution device 110b has read the ID information from the storage 100bA.

The function execution device 110b transmits the biological information on the user U and the ID information that have been acquired to the server 220 through the network 210. The server 220 causes the authenticator 124 to read the reference biological information from the storage based on the ID information. That is, the storage of the server 220 has the ID information and the reference biological information stored therein in an associated manner. The authenticator 124 of the server 220 extracts ID information matching with the ID information acquired from the function execution device 110b from ID information stored in the storage. The authenticator 124 of the server 220 reads the reference biological information associated with the extracted ID information. The authenticator 124 of the server 220 uses the same method as that of the authenticator 124 of the first embodiment to check for a match between the biological information on the user U and the read reference biological information to perform the authentication based on the biological information on the user U. The server 220 transmits the result of the authentication, that is, the result of the determination on whether to execute the predetermined function to the function execution device 110b. The function execution device 110b acquires the authentication result by the server 220, that is, the result of the determination on whether to execute the predetermined function, and causes the function controller 126 to execute the predetermined function based on the result of the determination. For example, the function execution device 110b makes payment processing through the card 100b as the predetermined function if the user U is determined to have been authenticated, or does not make payment processing through the card 100b if the user U is determined to have not been authenticated. The function execution device 110a may include the authenticator 124 and perform the authentication processing by itself without communicating with the server 220.

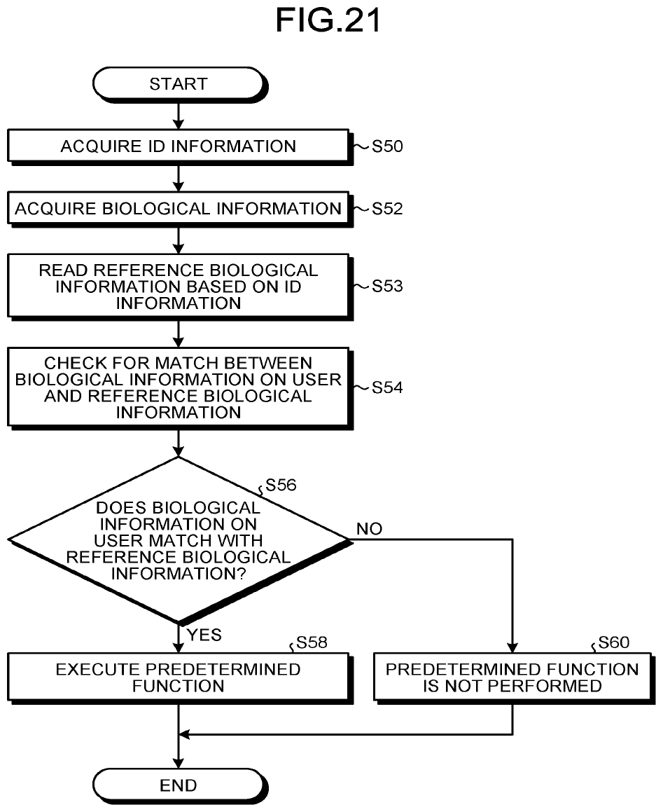

FIG. 21 is a flowchart explaining the authentication processing according to the third embodiment. As illustrated in FIG. 21, in the state where the card 100b is inserted in the function execution device 110b, the function execution device 110b reads the ID information stored in the storage 100bA of the card 100b (Step S50), and acquires the biological information on the user U detected by the sensor 10 of the card 100b (Step S52). The function execution device 110b transmits the ID information and the biological information that have been acquired to the server 220. The server 220 reads the reference biological information based on the ID information (Step S53), and checks for a match between the acquired biological information on the user U and the reference biological information (Step S54). The processing after Step S54 is the same as that after Step S14 in FIG. 15, and therefore will not be described.

As described above, the function execution device 110b acquires the biological information on the user U detected by the sensor 10, when triggered because the function execution device 110b has read the information (in this case, the ID information) on the card 100b serving as the portable object. Accordingly, the detection system 1 according to the present embodiment can execute the predetermined function at the time at which the predetermined function is required for the user U to be performed. As a result, the predetermined function can be executed at the appropriate time for the user U. Since the user need not perform an operation for the authentication to the function execution device 110a, the time and effort for the authentication can be reduced. The card is not limited to being configured to be inserted into the function execution device 110b, and may be configured to be held over (put in proximity to) the function execution device 110b. The sensor 10 may be provided with a light source for acquiring the biological information, and the light source may be started by power supplied from the function execution device. The light source may be provided in a position opposite to the sensor 10 with respect to the finger of the user, in an integrated manner with the function execution device, or in a position away from the function execution device. In this case, the wavelength of light of the light source emitted by, for example, the function execution device may be switched to that of, for example, the visible light or the infrared light depending on the biological information to be acquired.

The portable object may be a smartphone, a tablet computer, a smart watch, or a card including a storage for storing therein information on the user U. Such a portable object includes the sensor 10, and thereby, can reduce the time and effort for the authentication performed by the user U.

Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection system comprising:
a portable object that comprises a sensor configured to detect biological information on a user and is portable by the user; and
a function execution device configured to acquire the biological information on the user detected by the sensor through communication, and execute a predetermined function based on an authentication result of the user associated with the biological information on the user.

2. The detection system according to claim 1, wherein the function execution device is configured to acquire the biological information on the user detected by the sensor, when triggered because the portable object has been brought into a predetermined state.

3. The detection system according to claim 2, wherein the function execution device is configured to acquire the biological information on the user detected by the sensor, when triggered because the portable object and the function execution device are within a predetermined distance from each other.

4. The detection system according to claim 2, wherein the function execution device is configured to acquire the biological information on the user detected by the sensor, when triggered because the user has performed an operation for executing the predetermined function to the portable object or the function execution device.

5. The detection system according to claim 2, wherein the function execution device is configured to acquire the biological information on the user detected by the sensor, when triggered because the function execution device has read information on the portable object.

6. The detection system according to claim 1, wherein the portable object is a smartphone, a tablet computer, a smart watch, or a card comprising a storage configured to store therein information.

7. The detection system according to claim 1, wherein the sensor is configured to detect at least one of a vascular pattern of the user and a fingerprint of the user.

8. The detection system according to claim 7, wherein the sensor comprises a semiconductor containing amorphous silicon and a semiconductor containing polysilicon, and is configured to detect the vascular pattern of the user and the fingerprint of the user.

9. A method for authentication comprising:
a biological information acquiring step of acquiring biological information on a user through communication from a sensor that is provided in a portable object portable by the user, and is configured to detect the biological information on the user; and
a function executing step of executing a predetermined function based on an authentication result of the user associated with the biological information on the user.
